(19) United States
(12) Patent Application Publication
(10) Pub. No.: US 2019/0223770 A1
(43) Pub. Date: Jul. 25, 2019

(54) BIOLOGICAL SAMPLE EXTRACTION AND DETECTION SYSTEM

(71) Applicant: Bela Malik, Corpus Christi, TX (US)

(72) Inventor: Bela Malik, Corpus Christi, TX (US)

(21) Appl. No.: 15/879,258

(22) Filed: Jan. 24, 2018

(51) Int. Cl. A61B 5/145; A61B 5/00; A61B 5/1455; C12Q 1/6844; A61N 1/04

(52) U.S. Cl. CPC A61B 5/14546; A61B 5/4277; A61N 1/0412; C12Q 1/6844; A61B 5/1455

(57) ABSTRACT

An oral transmucosal-extraction device, system and components configured to be inserted or imbedded in an animal or human oral cavity for blood or interstitial fluid biological sample extraction, detection, and analysis. The device includes a receptacle for transmucosal-extraction, and is further configured to interface plurality of customizable function/s and application/s. The functions includes auxiliary body wearables, other medical devices, biologic and blood component detection, analysis, data collections, communication network, diagnostics for enhanced therapeutics and athletic performance. A part or entire device can also be configured to be inserted into a “reader,” which is external to the oral cavity. The reader is either “smart” or connected to a “smart device such as smart phone, and is configured to contain one or more sensors. The invention provides innovative information systems, and methods involving the aforedescribed devices, information available from blood, interstitial-fluids, biologics using transmucosal-extraction-sensor devices in the oral cavity and auxiliary devices.

### CROSS-REFERENCE TO RELATED APPLICATIONS

This application is continuation-in-part and claims priority to U.S. Pat. App. No. 62/180,841 entitled “Oral Biosensor Alerts and Communication System” which was filed on Jun. 17, 2015; U.S. patent application Ser. No. 14/850,713 entitled “Oral Sensor Alerting and Communication System and Developers' Tool Kit” which was filed on Sep. 10, 2015; U.S. patent application Ser. No. 15/836,901 entitled “In-situ Salivary Component Collection, Concentration, Isolation, Analytics, and Communication System” which was filed on Dec. 10, 2017, and which are incorporated herein. This application claims priority to U.S. Pat. App. No. 62/460,961 entitled “Biological Sample Extraction and Detection System” which was filed on Feb. 20, 2017.

### STATEMENT REGARDING FEDERALLY SPONSORED RESEARCH OR DEVELOPMENT

Not applicable.

The present disclosure relates generally to smart, implantable or attachable trans-mucosal blood or interstitial component extraction, collection, concentration, isolation, detection, and analysis devices; the integration of such with mobile communications, alerting and related technologies for both animals and humans, referred to herein as an BIOLOGICAL SAMPLE EXTRACTION AND DETECTION SYSTEM.

### BACKGROUND

Background is incorporated herein by reference from the U.S. patent application Ser. No. 14/850,713 entitled “Oral Sensor Alerting and Communication System and Developers' Tool Kit,” which was filed on Sep. 10, 2015, and paragraph numbers 004-006.

Oral cavity also has several characteristics that make it a great place for blood chemistry analysis via both blood and interstitial fluid analysis and biological detection. Mucosa of oral cavity has abundant blood supply and is fast wound healing without scaring, but, currently no system is available that utilizes the oral blood supply for determination of blood chemistry.

### SUMMARY OF THE INVENTION

The present invention provides smart oral cavity devices, systems and methods relating thereto, as well as auxiliary devices and methods, for greatly improving animal and human well-being through innovations in such technology. The invention combines its enhanced, trans-mucosal extraction device and smart oral sensors, diagnostic, and methods with communications, software management, data management, instant and long term animal and human analyses, multimedia inputs, visualizations, geometric motion, tracking, kinematics, alerting, therapeutic, electronic medical records, ophthalmoception, audioception, gustaoception, olfacoception or olfacception, tactioception, (thermoception), kinesthetic sense (proprioception), pain (nociception), balance (equilibrioception), vibration (mechanoreception), and various internal stimuli (e.g. the different chemoreceptors), tension sensors, pressure, stretch receptors, thirst, hunger, and time perception and other beneficial systems not previously available.

In both animals and humans, blood and interstitial fluids contain biologics, medication, nutritional molecules, etc. that are paramount in disease diagnostics and athletic performance. Blood and interstitial fluid analysis, commonly referred to as blood chemistry, includes, but not limited to, proteins, enzymes, pigments, lipids, gases, immunoglobulin, nucleic acids including modified nucleic acids, cations, anions, micronutrients, vitamins, minerals, allergens, salts, lactose, metabolites, cortisol, neuro-peptides, neuro-chemical, pH, hormones, lymph fluids, alpha-amylase, sugar, acetone, sulphur, alcohol, nicotine, electrolytes, blood borne pathogens, compounds, elements, and chemicals such as Ca²⁺, Na⁺, K⁺, H⁺, NaCl, phosphate, urea, uric acid, nitrogen, O²⁺, ammonia, uric acid, H₂O, glucose, bicarbonate, etc.

Several techniques used to extract blood chemicals via skin, called transdermal extraction, can also be used to do so through buccal and sublingual routes. “Mucosal extraction or transmucosal extraction” are terms coined here to represent extraction of blood and interstitial fluid components using technologies and methodologies. Mucosal extraction can have several benefits when compared to traditional methods used for blood and interstitial fluid analysis namely blood draw. Furthermore, oral tissues receive ample blood supply, so enough blood or interstitial components could be extracted and collected for accurate and consistent detection and analysis.

It is understood by anyone familiar with the arts that the device or parts thereof would be designed for most optimum contact and functioning through use of moldable, flexible, inflexible, fittable, moldable, adaptable, compliant, ductile, pliable, supple, tractable, tactile, transformable, workable, yielding, malleable, adjustable, changeable, conformable, convertible, bendable, and any combination thereof.

The device can be placed for sufficient amount of time to ensure proper measurements. Micro-pumps or suction micro-pump can enhance the interstitial fluid or blood component entry into the device. One or more micro-pump could be placed in any of the chambers that could assist in controlled manner suction of blood, interstitial fluid or its' components into various chambers.

Transmucosal extraction could either be coupled to sensors and form an oral sensor wearable technologies (OSWT)-transmucosal extraction system. OSWT-transmucosal extraction system of the invention provides for communication systems and alerting technology that links a multitude of transmucosal extraction information inputs together.

Besides serving as a receptacle for trans-mucosal extraction devices that can assist in blood and interstitial fluid extraction, the receptor could also contain collection, concentration, isolation module or chip for blood chemistry analysis via biosensor or sensor technologies. Sensors could monitor blood chemistry and biometrics through oral cavity or, at least, one or more module or chip of the said device could be removed for further processing, detection, and analysis by a medical device referred here as “reader” that could contain the sensors. The said device of the invention could entirely or partially which could include a module or chip could be implantable or attachable and de-attachable securely within a human or an animal's oral cavity.

It is understood by anyone familiar with arts that device with intra-oral cavity such as on dental devices and extra-oral cavity sensors such as in the aforementioned reader could be used interchangeably for all embodiments.

It is understood by anyone familiar with arts that the device could contain in-situ blood and interstitial component collection and concentration modules or chips. These chips could also be part of oral device and/or reader.

In a further embodiment, the invention provides where the “reader” contains one or more sensors and could also contain at least one interface with a network capable of utilizing the information obtained from the collected and concentrated salivary component and its interaction with at least one oral biosensor

Besides, being smart, the reader could also “read” biological information using sensors and/or accessory functions. In some instances, the light source on the cellular phone could be converted to excitation energy source; whereas, the camera could be converted to a light measuring device such as optical photo, density, or colorimeter, video, photodiode, LED. The camera could be used alone or in combination with other sensors to measure or record changes in biological activity. Furthermore, the touch sensors or pressure sensors in the cellular phone could be used to measure changes in volume, pressure, etc. Accessories could be attached to the cellular phone that contains sensors. The sensors could be selected from the group consisting of, but not limited to, contact, non-contact temperature, infrared, pressure of gas or liquid, absolute pressure differential pressure, vacuum pressure, gauge pressure, conductive rubber pressure, lead zirconate titanate pressure, Polyvinylidene fluoride pressure, PVDF-TrFE pressure, FETs pressure, metallic capacitive sensing elements pressure, resistance, tactile, elasto resistive sensors, conductivity, color, luminance, movement, optical, photo sensors, photo detectors, pixel (a light sensor and an active amplifier), light dependent resistors, optical filters, fluorescence, phosphorescence, sound, resonant, humidity, changes in humidity (measurements of mass, a mechanical, electrical changes as moisture is absorbed, could be used to measure humidity, or changes in temperature of condensation, changes in electrical capacitance, resistance, dielectric constant, dew point hygrometer, thermal conductivity temperature, or thermal conductivity), light, magnetic, electromagnetic, position, ionization, pH measurements, electrodes, fundamental electrical measurements, piezo-electric, piezo-resistive, potentiometric, orientation, video, 2 or 3D images, density, mass, MEMS, Lab-On-Chip to Micro Total Analysis, biosensors, chemo-sensors, biologic-sensor, a biologically-relevant-sensor, temperature, blood chemicals, blood electrolytes, pH, blood oxygen level, respired gases, gases, optimum breathing, oral air-flow, gyroscopic measurement, accelerometer measurement 1D, accelerometer measurement 2D, accelerometer measurement 3D, kinematics, ionic conductivity, photos, videos, images, electrical waves, sound waves, spectrophotometry, electromagnetic spectrum, gamma waves, X-ray wave, ultraviolet waves, visible waves, infrared waves, terahertz waves, microwaves, radio waves, magnetic waves, ultrasonic waves, magnetic resonance, magnetic field, electro- or magnetic-encephalography, functional magnetic resonance imaging, optical topography, global positioning or tracking, and radiation wave activity.

Both the oral device and reader could contain same or similar sensors and biosensors as described herein e.g., the sensors could be selected from the group consisting of, but not limited to, temperature, blood, pressure, teeth pressure, ionic conductivity, airflow, optimum breathing, oral air-flow, images, optical density, alterations to the oral cavity, surrounding muscle tone, muscle weakness, heart rate, heart rhythms, respiration rate, contact, non-contact temperature, infrared, pressure of gas or liquid, absolute pressure differential pressure, vacuum pressure, gauge pressure, conductive rubber pressure, lead zirconate titanate pressure, Polyvinylidene fluoride pressure, PVDF-TrFE pressure, FETs pressure, metallic capacitive sensing elements pressure, resistance, tactile, elasto resistive sensors, conductivity, color, luminance, movement, optical, photo sensors, photo detectors, pixel (a light sensor and an active amplifier), light dependent resistors, optical filters, fluorescence, phosphorescence, sound, resonant, humidity, changes in humidity (measurements of mass, a mechanical, electrical changes as moisture is absorbed, could be used to measure humidity, or changes in temperature of condensation, changes in electrical capacitance, resistance, dielectric constant, dew point hygrometer, thermal conductivity temperature, or thermal conductivity), light, magnetic, electromagnetic, position, ionization, pH measurements, electrodes, fundamental electrical measurements, piezo-electric, piezo-resistive, potentiometric, orientation, video, 2 or 3D images, density, mass, MEMS, Lab-On-Chip to Micro Total Analysis, biosensors, chemo-sensors, biologic-sensor, a biologically-relevant-sensor, temperature, blood chemicals, blood electrolytes, pH, blood oxygen level, respired gases, gases, optimum breathing, oral air-flow, gyroscopic measurement, accelerometer measurement 1D, accelerometer measurement 2D, accelerometer measurement 3D, kinematics, ionic conductivity, photos, videos, images, electrical waves, sound waves, spectrophotometry, electromagnetic spectrum, gamma waves, X-ray wave, ultraviolet waves, visible waves, infrared waves, terahertz waves, microwaves, radio waves, magnetic waves, ultrasonic waves, magnetic resonance, magnetic field, electro- or magnetic-encephalography, functional magnetic resonance imaging, optical topography, global positioning or tracking, and radiation wave activity.

In a further embodiment, the invention provides an blood or interstitial fluid collection, concentration, isolation and detection system wherein the system includes the above-described smart receptacle that contains at least one permeable membrane, semi-permeable membrane, micro-pump, paper microfluidics, milli-fluidics module, microfluidics module, nanofluidics chip or module, permeable membrane, semi-permeable membrane, non-permeable, synthetic membranes, biological membranes, coated membranes, uncoated membranes, color changing membranes, microfiltration membrane, ultra-filtration membranes, nano-filtration membranes, osmotic membranes, reverse osmotic membranes, ionic or charged membranes, neutral membranes, size-exclusion membranes, affinity membranes, adsorption membranes, extracting membranes, binding membranes, hydrophilic membranes, hydrophobic membranes, electrostatic attraction and repulsing membranes, polar membranes, non-polar membranes, lipophillic membranes, lipophobic membranes, coated membranes, resistant membranes, capillary action membranes, suction generating membranes, micro-pumps, absorbent materials, pressure generating membranes, surface tension generating membranes, energy releasing membranes, energy absorbing membranes, and reactive membranes. It is understood by anyone familiar with the arts that the membranes can be replaced by coated micro- or nano-beads, non-coated micro-, nano-beads or gel.

As aforementioned, several techniques are currently used for transdermal extractions including, but not limited to, electrical, thermal, vibrational or ultrasound, mechanically, chemical enhancers, and electro-chemical gradient assisted devices. Examples of electrically assisted are electroporation, ionophoresis, and reverse ionophororesis of thermally assisted are a laser, radio frequency heating, chemical heating, and mechanical heating. Ultrasound assisted are sonophoresis and phonophoresis; mechanical assisted are micro-needles and tape-stripping; chemical and electro-chemical gradient based are salt, natural, man-made polymers and compound assisted diffusion, surfactants, enzymes, salts, chemical- or enzyme-peels, nanoparticles, polymer-chain, microparticulate, etc. These devices can be miniaturized to fit in the oral cavity as are dermal patches, and hence, can be currently manufactured in large customizable quantities by use of mico-, nano-electromechanics, micro- or nano-fluidics, 3D printing, etc.

The dental devices could contain OSWT-transmucosal extraction or transmucosal extraction devices that can assist in blood and interstitial extraction is selected from the group consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible trans-mucosal extraction patch, stretch stripes, adhesive stripes, trans-mucosal extraction patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a trans-mucosal extraction gauge, trans-mucosal extraction dipstick, trans-mucosal extraction rod, trans-mucosal extraction stick, biteplate, bendable or flexible trans-mucosal extraction device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent trans-mucosal extraction and electronic for analytical and other functions and applications in order to provide accurate physiological information. These extraction systems and sensors are used so that they cannot be swallowed and the trans-mucosal extraction device is in contact with oral tissues for extraction to occur.

The mucosal extraction devices could be electrical, thermal, vibrational or ultrasound, mechanically, chemical enhancers, and velocity assisted devices. Examples of electrically assisted are electroporation and ionophoresis, of thermally assisted are a laser, radio frequency heating, chemical heating, and mechanical heating. Ultrasound assisted is sonophoresis and phonophoresis; mechanical assisted are micro-needles and tape-stripping. Chemical and electro-chemical gradient based are salt, natural, man-made polymers and compound assisted diffusion, inhibitors, surfactants, enzymes, salts, chemical- or enzyme-peels, liposomes, enzyme inhibitors, nanoparticles, polymer-chain, microparticulate, mucoadhesives, diffusion, osmosis, etc.

OSWT-transmucosal extraction or transmucosal extraction system of the invention provides for communication systems and alerting technology that links a multitude of transmucosal extraction information inputs together. This method of gathering diagnostic and biometric information and integration of transmucosal extraction from oral devices provides the basis for a real-time or near-time or long-term snapshot of effects of medication on an animal or human's health and well-being. This system could be used with the aforementioned reader containing sensors where OSWT could be replaced by a sensor in the reader. In this application, embodiments could use either OSWT-transmucosal extraction system or transmucosal extraction system. However, when the reader is coupled to transmucosal extraction system is used, real-time alerts would be replaced by near-time alerts.

In one embodiment of the invention, one or more devices that assist mucosal extraction in the oral cavity can be part of or attached to a dental device such that the blood chemistry or interstitial fluid can be effectively analyzed. For example, the module containing an array of micro-needles containing electrodes could be easily attached and de-attached to a dental device. Micro-needles are multiple microscopic projections typically assembled on one side of a supporting base or patch, generally ranging from 25 to 2000 μm in height, 50 to 250 μm in base width and 1 to 25 μm in tip diameter. These micro-electrodes could assist in the collection of blood and interstitial fluids for measurements and analysis of dehydration levels, cations, anions, salts, etc. The device can be placed for sufficient short or long amount of time to ensure analysis. Additionally, to achieve maximum results more than one transmucosal extraction methodologies can be used together.

In one embodiment of the invention, reverse iontophoresis technology can be used to diagnose and monitoring blood glucose levels. One major advantage of iontophoresis in diagnostic applications is that there is no mechanical penetration or disruption of the mucosal lining, and thus, it is non-invasive. Iontophoresis involves the application of physiologically acceptable electrical currents (about 0.1-1.0 mA/cm²) to drive charged molecules across the mucosal lining by electrostatic effects, and thus, biological molecules can pass through the mucosa into the device due to the potential gradient. This device can be coupled to a drug dispensing pump in the oral cavity, or another part of the body, or send out alerts to dispense medications.

Laser ablations can also be used to extract interstitial fluid for subsequent measurement of glucose levels in diabetic patients. In this device, the degree of barrier disruption achieved is controlled by wavelength, pulse length, tissue thickness, pulse energy, tissue absorption coefficient, pulse number, duration of laser exposure and pulse repetition rate. Low-frequency ultrasound can also be applied to extract interstitial fluid across the mucosal lining. Radiofrequency (RF) thermal ablation is performed by the placement of a thin, needle-like electrode directly into the mucosa and application of high-frequency alternating current (˜100 kHz). A variety of analytes including glucose, albumin, calcium, urea, uric acid, triglycerides, lactate, and dextran can be transmucosal extracted using these approaches. The composition of the fluid extracted trans-mucosally from oral tissues would be similar to that of blood or interstitial fluid.

Accordingly, oral sensors and transmucosal extraction device alerts and communication system, methods and devices related to and used in conjunction therewith are provided which address the needs and provide the advantages outlined herein.

Also provided is an OSWT-transmucosal extraction device according to the invention where transmucosal extraction system and oral sensors and other sensors and devices are connected to “smart” medical devices in response to alerts and/or signals from the OSWT-transmucosal extraction system.

In an aspect of the invention, a device is provided which includes a smart transmucosal extraction and, perhaps, sensor receptacle for the oral device. The receptacle is configured to be inserted in an oral cavity of an animal or human. The receptacle is configured to serve one or more functions within the animal or human's oral cavity wherein the one or more functions could be customized by physicians, veterinarians, patients, animal owners, users, and caretakers. The customizable functions can utilize mechatronics and can be integrated with transmucosal extraction devices and sensors selected to treat, medicate, measure, and diagnose one or more medical health and therapeutic biometrics. OSWT system imbedded oral cavity sensors and transmucosal extraction devices could be utilized in conjunction with dental and oral devices listed elsewhere in this application. The device includes one or more medication delivery, dispensing, and administration systems and sensors contained within or upon the receptacle or multiple receptacles in the oral cavity or outside of the oral cavity.

In another embodiment of the invention, the OSWT-transmucosal extraction system can streamline and integrate performance measurements such as, but not limited to, various geometric models, visualization, complex spatial-temporal relations, human and animal facial and physical relationships (individually and group), data associations (i.e., pixels, auditory, motion, optimum breathing, oral air-flow, accelerometers, gyroscope, metabolic biosensors, transmucosal extraction devices, high-definition video capture, body-wearable sensors and transmucosal extraction devices, RFIDs, readers, positioning, micro- and nano-electronics, micro- and nano-enabled energy harvesting, micro- and nano-energy storage, micro- and nano-devices, micro- and nano-timer, micro- and nano-devices, micro- and nano-programmable processors, micro- and nano-memory, micro- and nano-integrated power management, micro- and nano-programmable hardware, micro- and nano-wireless communication capabilities across multiple, various degrees of dynamic alerting, tracking, positioning, multi-media, analytics, historical and other comparative data inputs, communications and platforms). Collectively, these inputs can be synced and integrated with all forms of data capture. The OSWT-transmucosal extraction system can provide important real-time or near time analytics to correct or modify a motion, behavior for individuals, or organizational groups for animals and humans.

In a further embodiment, the invention provides an OSWT-transmucosal extraction system including the above-described smart receptacle, one or more active medication delivery and dispenser systems and sensors contained within, attached, or upon the receptacle and at least one interface with a network configured to utilize the information obtained from the one or more dispensers and sensors.

It is understood by anyone familiar with the art that independent of wireless storage, the data could be stored in any OSWT-transmucosal extraction device through any digital storage device, connector, or mechanism.

In yet a further embodiment of the invention, a method is provided for obtaining sensors and transmucosal extraction devices' data from a human and/or an animal. The device includes smart sensors and transmucosal extraction devices receptacle configured to serve one or more functions within a human or an animal's oral cavity. The smart receptacle contains or receives within or upon it one or more sensors and transmucosal extraction devices capable of providing information relevant to the health, therapeutic, or a physiological characteristic of the human or animal. The device further involves activating or monitoring the one or more sensors and transmucosal extraction devices to obtain or analyze the information relevant to the health, therapeutic, or a physiological characteristic of the human or animal and transmitting at least some portion of the health, therapeutic, or physiological information or analysis to a network capable of utilizing the information obtained. The one or more functions of the transmucosal extraction devices receptacle is configured to serve, e.g., is selected from the group consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible trans-mucosal extraction patch, stretch stripes, adhesive stripes, trans-mucosal extraction patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a trans-mucosal extraction gauge, trans-mucosal extraction dipstick, trans-mucosal extraction rod, trans-mucosal extraction stick, biteplate, bendable or flexible trans-mucosal extraction device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent trans-mucosal extraction and electronic for analytical and other functions and applications in order to provide accurate physiological information. These extraction systems and sensors are used so that they cannot be swall owed.

It is understood by anyone familiar with the arts that the transmucosal extraction device could contain software for the device functionality including but not limited to alert system, wearable networking, one or more electrical input; one or more transducers configured to convert input electrical energy to different energy forms for activating and implementing extraction procedure; a controller configured for providing the control signals, intensity, duration, timing, sequence, and nature information for the different energies supplied to the device said transducers; and one or more compartment configured for either collection of extracted fluids and/or detecting the blood and interstitial components.

The recognition component in these systems and methods of the invention, often called a receptor, can use, e.g., biomolecules from organisms or receptors modeled after biological systems to interact with an analyte of interest. This interaction can be measured by a bio-transducer which outputs a measurable signal proportional to the presence of a target analyte in the sample.

In yet an additional aspect, the invention includes a diagnostic or other systems for an animal or human. The diagnostic or other system includes a smart, wearable or attachable device, or a device insertable to the body or other component, internal and/or external to the oral cavity. The system also includes a receptacle configured for placement within the oral cavity of the animal or human with one or more functions of the transmucosal extraction devices receptacle is configured to serve, e.g., is selected from the group consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible trans-mucosal extraction patch, stretch stripes, adhesive stripes, trans-mucosal extraction patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a trans-mucosal extraction gauge, trans-mucosal extraction dipstick, trans-mucosal extraction rod, trans-mucosal extraction stick, biteplate, bendable or flexible trans-mucosal extraction device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent trans-mucosal extraction and electronic for analytical and other functions and applications in order to provide accurate physiological information. The system also includes one or more sensors and transmucosal extraction devices located within or upon the oral cavity receptacle. The smart, wearable, attachable or externally insertable device is configured to obtain information from, provide information to, or both, the one or more sensors and transmucosal extraction devices located within or upon the oral cavity receptacle. And, the one or more sensors and transmucosal extraction devices or the smart, external device, or both, are configured to transmit the information to a network.

Also provided is a customizable development tool kit or platform for multiple OSWT-transmucosal extraction purposes and functions and for building a diagnostic or other system to provide information such as, but not limited to hardware, and software integration, analysis, or alerts, for an animal, animals, human or humans. The kit includes customizable components to meet the needs of a consumer or user of the diagnostic or other system. The components include at least one transmucosal extraction devices with or without sensor insertable or temporarily or permanently imbedded within or upon one or more oral cavity receptacles, and at least one oral cavity receptacle configured for placement within the oral cavity of the animal or humans, at least one smart sensor and auxiliary device which is wearable, attachable or insertable externally to the oral cavity. The tool kit also includes at least one network unit configured to receive information from the at least one smart, auxiliary device, the at least one sensors and transmucosal extraction devices, or both, and analyze, transmit or both, the information received. The components for selecting the at least one auxiliary device, the at least one oral cavity transmucosal extraction devices with or with sensor receptacle, the at least one transmucosal extraction devices, and the at least one network unit are made available to the consumer or user to construct or have constructed a diagnostic or transmucosal extraction or other system configured to obtain information, analysis or alerts customized to meet the specific needs of the consumer or user.

Oral mucosal transmucosal extraction can have several benefits when compared to traditional blood drawn, injections, transdermal routes, and others. Anyone familiar with the arts understands that transmucosal extraction through oral mucosa non-invasive, decreased pain and increased patient compliance, decreased generation of dangerous medical sharps, and reduced risk of needle contamination, disease transmission, and needle misuse and lower cost. Transmucosal extraction can allow for quick or sustained diagnostic and biometric measurements from both systemic and localized areas. These could be painless, cause little or no bleeding, eliminate possibility of severe rash and skin infections, minimal possibility of pathogens introduction, can be self-administration, and reduce instances of accidental needle-sticks injuries. Transmucosal extraction could allow enough blood and interstitial fluid component extraction for accurate and consistent detection, which is a major problem with transdermal extraction systems. The transmucosal extraction device could be connected to a secure network for plurality of functions.

### BRIEF DESCRIPTION OF THE DRAWINGS

The presently disclosed subject matter will be better understood from reading the following description of non-limiting embodiments, with reference to the attached drawings, wherein below:

FIG. 1 is a schematic depiction which exemplifies an OSWT-transmucosal extraction and transmucosal extraction device system secure wireless communication capability and information retrieval through the animal's or human's oral cavity according to the invention.

FIG. 2 is a schematic depiction showing which oral cavity tissue or portions could be most useful for diagnostic measurements. Here we show a diagram with different parts of oral cavity. Some parts are better for transmucosal extraction than others parts of the oral cavity.

FIG. 3 is a representative of cellular structure of oral mucosa: The oral mucosa is of two types, keratinized and non-keratinized. Areas that are subjected to mechanical stress are keratinized and are similar to the epidermis of the skin. Areas that are not subjected mechanical stress are not keratinized and unlike skin form less barrier for diagnostic measurements.

FIG. 4 depicts a schematics depiction that exemplifies the structure of a transmucosal extraction device along with the sensors or biosensors, forming OSWT-transmucosal extraction system, as may be used in embodiments of the invention.

FIG. 5 depicts embodiments for various passive and active technologies to enhance diagnostic measurements through the oral cavity.

FIG. 6 depicts embodiments which exemplify OSWT-transmucosal extraction or transmucosal extraction system dynamic alerting software and secure networks, in accordance with embodiments of the present invention.

### DETAILED DESCRIPTION OF THE INVENTION

The foregoing summary, as well as the following detailed description of certain embodiments will be better understood when read in conjunction with the appended drawings. As used herein, an element or step recited in the singular and proceeded with the word “a” or “an” should be understood as not excluding the plural of said elements or steps, unless such exclusion is explicitly stated. Furthermore, references to “one embodiment” or “an embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

As used herein, the term “smart” means a device or object that performs one or more functions of a computer or information system, such as data storage, calculation, Internet access and information transmission.

As used herein the terms “insertable”, “implantable”, “imbeddable”, “embeddable”, “temporarily insertable” “permanently insertable”, “temporarily implantable”, “permanently implantable”, “temporarily imbeddable”, “permanently imbeddable”, “temporarily embeddable” and “permanently embeddable” refer to means of securely inserting and attaching in or to, or fastening a device, such as being adhered to, cemented, affixed or otherwise securely attached to a surface or object.

As used herein, the term “receptacle” refers to a device or container that receives, retains, has within, or holds something.

“Mucosal extraction or transmucosal extraction” are terms coined here to represent extraction of blood and interstitial fluid components using extraction technologies and methodologies, can also be used to do so through buccal and sublingual routes.

Described in its broader respects, the Oral Sensors and transmucosal extraction device Alerts and Communication Transmucosal Extraction (OSWT-Transmucosal Extraction) system of the invention includes a device configured to be inserted and securely attached in an oral cavity of an animal or human. The device includes a transmucosal extraction and smart sensor receptacle for a sensors and diagnostic enhancement device for blood and interstitial fluid analysis.

Blood chemistry and interstitial fluid analysis includes, but not limited to, proteins, enzymes, pigments, lipids, gases, immunoglobulin, nucleic acids including modified nucleic acids, cations, anions, micronutrients, vitamins, minerals, allergens, salts, lactose, metabolites, cortisol, neuro-peptides, neuro-chemical, pH, hormones, lymph fluids, alpha-amylase, sugar, acetone, sulphur, alcohol, nicotine, electrolytes, blood borne pathogens, compounds, elements, and chemicals such as Ca²⁺, Na⁺, K⁺, H⁺, NaCl, phosphate, urea, uric acid, nitrogen, O²⁺, ammonia, uric acid, H₂O, glucose, bicarbonate or CO₂. Term “biometrics” in this application refers to measurements of these and other blood components and vital symptoms.

The receptacle is preferably configured to serve one or more functions within the animal or human's oral cavity without being swallowed. The device also includes transmucosal extraction device with or without one or more sensors contained within or upon the receptacle, and also at least one interface with a network configured to utilize the information obtained from the one or more sensors and transmucosal extraction device or from one or more platforms providing additional information or capabilities networked with the system. These devices can be miniaturized to fit in the oral cavity, and hence, can be currently manufactured in large customizable quantities by use of mico-, nano-electromechanics, micro- or nano-fluidics, 3D printing, etc.

It is understood by anyone familiar with the arts that the device or parts thereof would be designed for most optimum contact and functioning through use of moldable, flexible, inflexible, fittable, moldable, adaptable, compliant, ductile, pliable, supple, tractable, tactile, transformable, workable, yielding, malleable, adjustable, changeable, conformable, convertible, bendable, and any combination thereof.

Transmucosal extraction devices can be part of dental or oral devices these include or can be selected from the group consisting of one or more functions of the transmucosal extraction devices receptacle is configured to serve, e.g., is selected from the group consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible trans-mucosal extraction patch, stretch stripes, adhesive stripes, trans-mucosal extraction patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a trans-mucosal extraction gauge, trans-mucosal extraction dipstick, trans-mucosal extraction rod, trans-mucosal extraction stick, biteplate, bendable or flexible trans-mucosal extraction device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent trans-mucosal extraction and electronic for analytical and other functions and applications in order to provide accurate physiological information. These extraction systems and sensors are used so that they cannot be swallowed.

Furthermore, this system could includes a device insertion and release mechanism which is capable of being mechanically and electrically connected to and disconnected from the OSWT-Transmucosal Extraction or Transmucosal Extraction devices these include or can be selected receptacle is configured to serve, e.g., is selected from the group consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible trans-mucosal extraction patch, stretch stripes, adhesive stripes, trans-mucosal extraction patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a trans-mucosal extraction gauge, trans-mucosal extraction dipstick, trans-mucosal extraction rod, trans-mucosal extraction stick, biteplate, bendable or flexible trans-mucosal extraction device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent trans-mucosal extraction and electronic for analytical and other functions and applications in order to provide accurate physiological information. These extraction systems and sensors are used so that they cannot be swall owed.

These dental devices could contain transmucosal extraction device, which could be electrical, thermal, vibrational or ultrasound, mechanically, chemical enhancers, and velocity assisted devices. Examples of electrically assisted are electroporation, ionophoresis, and reverse ionophoresis, of thermally assisted are a laser, radio frequency heating, chemical heating, and mechanical heating. Ultrasound assisted are sonophoresis and phonophoresis; mechanical assisted are micro-needles and tape-stripping; Chemical and electro-chemical gradient based are salt, natural, man-made polymers and compound assisted diffusion, inhibitors, surfactants, enzymes, salts, chemical- or enzyme-peels, liposomes, enzyme inhibitors, nanoparticles, polymer-chain, microparticulate, mucoadhesives, diffusion, osmosis, etc. These devices can be miniaturized to fit in the oral cavity by use of mico-electronics and nanotechology.

Oral mucosal transmucosal extraction could have several benefits when compared to traditional injections, blood drawn. Anyone familiar with the arts understands that advantages of transmucosal extraction over traditional blood drawn include, non-invasive, decreased pain and increased patient compliance, decreased generation of dangerous medical sharps, and reduced risk of needle contamination, disease transmission, and needle misuse. Oral mucosal extraction could allow for quick or sustained profiles measurements for both systemic and localized transmucosal extraction. Mucosal extraction could provide enough blood or interstitial component for accurate and consistent detection and analysis. Several techniques used to measure blood and interstitial fluid chemicals to diagnose can be used for buccal and sublingual routes.

In one embodiment of the invention, one or more devices that assist transmucosal extraction in the oral cavity can be part of or attached to a dental device such that the blood or interstitial fluid component measurements can be effectively done. The array contains the transmucosal device places at a location within the oral cavity such that maximum transmucosal extraction could occur. The device can be placed for sufficient amount of time to ensure proper measurements. Micro-pumps or suction micro-pump can enhance the interstitial fluid or blood component entry into the device. One or more micro-pump could be placed in any of the chambers that could assist in controlled manner suction of saliva or its' components into various chambers.

In a further embodiment, the invention provides an blood or interstitial fluid collection, concentration, isolation and detection system wherein the system includes the above-described smart receptacle that contains at least one permeable membrane, semi-permeable membrane, micro-pump, paper microfluidics, milli-fluidics module, microfluidics module, nanofluidics chip or module, permeable membrane, semi-permeable membrane, non-permeable, synthetic membranes, biological membranes, coated membranes, uncoated membranes, color changing membranes, microfiltration membrane, ultra-filtration membranes, nano-filtration membranes, osmotic membranes, reverse osmotic membranes, ionic or charged membranes, neutral membranes, size-exclusion membranes, affinity membranes, adsorption membranes, extracting membranes, binding membranes, hydrophilic membranes, hydrophobic membranes, electrostatic attraction and repulsing membranes, polar membranes, non-polar membranes, lipophillic membranes, lipophobic membranes, coated membranes, resistant membranes, capillary action membranes, suction generating membranes, micro-pumps, absorbent materials, pressure generating membranes, surface tension generating membranes, energy releasing membranes, energy absorbing membranes, and reactive membranes. It is understood by anyone familiar with the arts that the membranes can be replaced by coated micro- or nano-beads, non-coated micro-, nano-beads or gel.

Besides serving as a receptacle for trans-mucosal extraction devices that could assist in blood and interstitial fluid extraction, the receptor could also contain collection and concentration module or chip for blood chemistry analysis via sensor technologies. Sensors could monitor blood chemistry and biometrics through oral cavity or, at least, one or more module or chip of the said device could be removed for further processing, detection, and analysis by a medical device referred here as “reader” that could contain the sensors. The said device of the invention could include a module or chip which is implantable or attachable and de-attachable securely within a human or an animal's oral cavity.

In a further embodiment, the invention provides where the “reader” contains one or more sensors and could also contain at least one interface with a network capable of utilizing the information obtained from the collected and concentrated salivary component and its interaction with at least one oral biosensor

Besides, being smart, the reader could also “read” biological information using sensors and/or accessory functions. In some instances, the light source on the cellular phone could be converted to excitation energy source; whereas, the camera could be converted to a light measuring device such as optical photo, density, or colorimeter, video, photodiode, LED. The camera could be used alone or in combination with other sensors to measure or record changes in biological activity. Furthermore, the touch sensors or pressure sensors in the cellular phone could be used to measure changes in volume, pressure, etc. Accessories could be attached to the cellular phone that contains sensors. The sensors could be selected from the group consisting of, but not limited to, contact, non-contact temperature, infrared, pressure of gas or liquid, absolute pressure differential pressure, vacuum pressure, gauge pressure, conductive rubber pressure, lead zirconate titanate pressure, Polyvinylidene fluoride pressure, PVDF-TrFE pressure, FETs pressure, metallic capacitive sensing elements pressure, resistance, tactile, elasto resistive sensors, conductivity, color, luminance, movement, optical, photo sensors, photo detectors, pixel (a light sensor and an active amplifier), light dependent resistors, optical filters, fluorescence, phosphorescence, sound, resonant, humidity, changes in humidity (measurements of mass, a mechanical, electrical changes as moisture is absorbed, could be used to measure humidity, or changes in temperature of condensation, changes in electrical capacitance, resistance, dielectric constant, dew point hygrometer, thermal conductivity temperature, or thermal conductivity), light, magnetic, electromagnetic, position, ionization, pH measurements, electrodes, fundamental electrical measurements, piezo-electric, piezo-resistive, potentiometric, orientation, video, 2 or 3D images, density, mass, MEMS, Lab-On-Chip to Micro Total Analysis, biosensors, chemo-sensors, biologic-sensor, a biologically-relevant-sensor, temperature, blood chemicals, blood electrolytes, pH, blood oxygen level, respired gases, gases, optimum breathing, oral air-flow, gyroscopic measurement, accelerometer measurement 1D, accelerometer measurement 2D, accelerometer measurement 3D, kinematics, ionic conductivity, photos, videos, images, electrical waves, sound waves, spectrophotometry, electromagnetic spectrum, gamma waves, X-ray wave, ultraviolet waves, visible waves, infrared waves, terahertz waves, microwaves, radio waves, magnetic waves, ultrasonic waves, magnetic resonance, magnetic field, electro- or magnetic-encephalography, functional magnetic resonance imaging, optical topography, global positioning or tracking, and radiation wave activity.

Both the oral device and reader could contain same or similar sensors and biosensors as described herein e.g., the sensors could be selected from the group consisting of, but not limited to, temperature, blood, pressure, teeth pressure, ionic conductivity, airflow, optimum breathing, oral air-flow, images, optical density, alterations to the oral cavity, surrounding muscle tone, muscle weakness, heart rate, heart rhythms, respiration rate, contact, non-contact temperature, infrared, pressure of gas or liquid, absolute pressure differential pressure, vacuum pressure, gauge pressure, conductive rubber pressure, lead zirconate titanate pressure, Polyvinylidene fluoride pressure, PVDF-TrFE pressure, FETs pressure, metallic capacitive sensing elements pressure, resistance, tactile, elasto resistive sensors, conductivity, color, luminance, movement, optical, photo sensors, photo detectors, pixel (a light sensor and an active amplifier), light dependent resistors, optical filters, fluorescence, phosphorescence, sound, resonant, humidity, changes in humidity (measurements of mass, a mechanical, electrical changes as moisture is absorbed, could be used to measure humidity, or changes in temperature of condensation, changes in electrical capacitance, resistance, dielectric constant, dew point hygrometer, thermal conductivity temperature, or thermal conductivity), light, magnetic, electromagnetic, position, ionization, pH measurements, electrodes, fundamental electrical measurements, piezo-electric, piezo-resistive, potentiometric, orientation, video, 2 or 3D images, density, mass, MEMS, Lab-On-Chip to Micro Total Analysis, biosensors, chemo-sensors, biologic-sensor, a biologically-relevant-sensor, temperature, blood chemicals, blood electrolytes, pH, blood oxygen level, respired gases, gases, optimum breathing, oral air-flow, gyroscopic measurement, accelerometer measurement 1D, accelerometer measurement 2D, accelerometer measurement 3D, kinematics, ionic conductivity, photos, videos, images, electrical waves, sound waves, spectrophotometry, electromagnetic spectrum, gamma waves, X-ray wave, ultraviolet waves, visible waves, infrared waves, terahertz waves, microwaves, radio waves, magnetic waves, ultrasonic waves, magnetic resonance, magnetic field, electro- or magnetic-encephalography, functional magnetic resonance imaging, optical topography, global positioning or tracking, and radiation wave activity.

Transmucosal extraction could either be coupled to sensors and form an OSWT-transmucosal extraction system. OSWT-transmucosal extraction system of the invention provides for communication systems and alerting technology that links a multitude of transmucosal extraction information inputs together. This method of gathering diagnostic and biometric information and integration of transmucosal extraction from oral devices provides the basis for a real-time or near-time or long-term snapshot of effects of medication on an animal or human's health and well-being. This system could be used with the aforementioned reader containing sensors where OSWT could be replaced by a sensor in the “reader.” In this application, embodiments could use either OSWT-transmucosal extraction system or transmucosal extraction system. However, when the reader is coupled to transmucosal extraction system is used, real-time alerts would be replaced by near-time alerts.

It is understood by anyone familiar with arts that device with intra-oral cavity such as on dental devices and extra-oral cavity sensors such as in the aforementioned reader could be used interchangeably for all embodiments.

It is understood by anyone familiar with arts that the device could contain in-situ blood and interstitial component collection and concentration modules or chips. These chips could also be part of oral device and/or reader.

Some transmucosal extraction systems and/or sensors can also be placed on medical devices used during surgery and hospitalization. These extraction systems and sensors are used so that they cannot be swallowed and the transmucosal extraction device is always in contact with oral tissues for extraction to occur.

It is understood by anyone familiar with the arts that the transmucosal extraction device could contain software for the device functionality including but not limited to alert system, wearable networking, etc., one or more electrical input; one or more transducers configured to convert input electrical energy to different energy forms for activating and implementation of extraction procedure; a controller configured for providing the control signals, amplitude, intensity, optimized pulse, duration, timing, sequence, simultaneous, and/or nature information for the different energies supplied to the device said transducers; and one or more compartment configured for either collection of extracted fluids and/or detecting the blood and interstitial components. Furthermore, the controller could be a microprocessor with related and relevant hardware, electronics, and software configured for controlling the different factors comprising of temperature, rate of heating, duration of heating, the ultrasonic energy, frequency, amplitude, intensity, and its duration for detection and analysis of analytes in blood and interstitial fluids and displaying, storage, or communication of collected data using a program for accurate and efficient detection. This system includes a device insertion and release mechanism which is capable of being mechanically and electrically connected to and disconnected from the controller. Furthermore, the controller includes a memory for the storage, recording and retrieval of data for further use and also capable of connecting and data sharing to the external devices via wire and/or wirelessly means.

In one embodiment of the invention, the input power source specifically could be a battery, micro- or nano-power generators or storage, an AC power adapter and/or a capacitor. Wherein the input power source could be configured for delivering a current of range 0.001 mA-1000 mA, and the input power source could be configured for a potential delivery range of about 0 to 15 V DC. Furthermore, the device could comprises of a plurality of circuit boards and blocks which are configured and connected to one or more transducers for converting the input electrical energy to heat, ultrasonic vibration or energy via ultrasonic transducer/piezoelectric device configured for delivering ultrasonic vibrations with a frequency range of about 18 KHz-30 KHz, electromotive force, etc such that the resistive pattern could be configured for heating the oral tissue and/or the device to a temperature of at least 20-200° C. These circuit boards and blocks could use one or more conductive electrodes to deliver a current of range 0.001 mA-100 mA and act as conduit for transferring electromotive force to the oral tissue. These circuit boards and blocks could be reusable and could be part of and/or integrated with the controller.

In another embodiment of the invention, the trans-mucosal extraction device could be turned on or off either manually by the user or care-taker or could be turned on or off automatically by use of a pre-programmed software.

In an aspect of the invention, a device is provided which includes a smart transmucosal extraction and, perhaps, sensor receptacle for the oral device. The receptacle is configured to be inserted in an oral cavity of an animal or human. The receptacle is configured to serve one or more functions within the animal or human's oral cavity wherein the one or more functions could be customized by physicians, veterinarians, patients, animal owners, users, and caretakers. The customizable functions can utilize mechatronics and can be integrated with transmucosal extraction devices and sensors selected to treat, medicate, measure, and diagnose one or more medical health and athletic performance biometrics. OSWT system imbedded oral transmucosal extraction devices and sensors could be utilized in conjunction with including, but not limited to, is selected from the group consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible trans-mucosal extraction patch, stretch stripes, adhesive stripes, trans-mucosal extraction patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a trans-mucosal extraction gauge, trans-mucosal extraction dipstick, trans-mucosal extraction rod, trans-mucosal extraction stick, biteplate, bendable or flexible trans-mucosal extraction device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent trans-mucosal extraction and electronic for analytical and other functions and applications in order to provide accurate physiological information. The device includes one or more medication delivery, dispensing, and administration systems and sensors contained within or upon the receptacle or multiple receptacles, but these devices would be conferred such that they cannot be swallowed.

In a further embodiment, the invention provides an OSWT-transmucosal extraction system including the above-described smart receptacle, one or more active medication delivery and dispenser systems and sensors contained within, attached, or upon the receptacle and at least one interface with a network configured to utilize the information obtained from the one or more dispensers and sensors.

It is understood by anyone familiar with the art that independent of wireless storage, the data could be stored in any OSWT-transmucosal extraction device through any digital storage device, connector, or mechanism.

In addition, the OSWT-transmucosal extraction system of the invention includes storing secure data captured by the oral cavity when no connectivity is available. The OSWT-transmucosal extraction system of the invention both stores and/or stream data and/or packeted as programmed. In addition, if OSWT-transmucosal extraction-captured data is securely stored within the mouth cavity or in close proximity to the biosensors and transmucosal extraction devices, it is optionally streamed when connectivity becomes available. In addition, audio, video or any form of enriched data (multimedia) is activated when necessary for security purposes and protection. The activation of both biosensors and transmucosal extraction devices and data capture (camera, mic, etc.) is optionally hidden within the oral cavity and/or is activated in a stealth manner, using techniques known to those familiar with the art.

The system as described above may provide, e.g., one or more functions of the device including or selected from the group consisting of one or more functions of the transmucosal extraction devices receptacle is configured to serve, e.g., is selected from the group consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible trans-mucosal extraction patch, stretch stripes, adhesive stripes, trans-mucosal extraction patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a trans-mucosal extraction gauge, trans-mucosal extraction dipstick, trans-mucosal extraction rod, trans-mucosal extraction stick, biteplate, bendable or flexible trans-mucosal extraction device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent trans-mucosal extraction and electronic for analytical and other functions and applications in order to provide accurate physiological information. These transmucosal extraction devices could be integrated to body sensors, health-devices, nano-particles, drug delivery systems, and sports and performance sensors and sensors on inanimate objects and sports equipment; customizable developers' tool kit for biosensors, performance, medical analytics, oral and systemic body diagnosis; integrated, pre-integrated and post-integrated, platforms; any type of medium, secure bidirectional media, multiple media, video, audio, 3D, printing, reporting, analytics, reporting, metadata diagnosis, with geometric tracking, communication networks, analytics, alerting, kinematics for individuals, team sports, organizational groups, animals and humans, communications, software management, data management, instant and long term animal and human analyses, multimedia inputs, visualizations, geometric motion, tracking, kinematics, alerting, therapeutic, electronic medical records, historical analysis, time stamped data, reporting and feedback, positioning, the integrated video can be synced with all wearables and other biosensors and transmucosal extraction devices in order to produce computer-generated precise movement and greater precision or analytics.

Additionally, the system may further include transmucosal extraction device and one or more sensors and contained within or upon one or more receptacles located within or upon the animal or human's body networked with the oral cavity device.

Also provided is an OSWT-transmucosal extraction device according to the invention where transmucosal extraction system and oral sensors and other sensors and devices are connected to “smart” medical devices in response to alerts and/or signals from the OSWT-transmucosal extraction system. One example, of this, could be used as a closed-loop insulin delivery system. A closed-loop insulin delivery system is essentially an artificial pancreas where the loop refers to the continuous cycle of feedback information. As the blood glucose level change continuously, the change is detected by the transmucosal extraction smart sensor system, and the information is continuously sent to the insulin pump, which adjusts its insulin output; and the blood glucose level changes again in response to the insulin. The loop is closed when this happens automatically and semi-closed when changes are made by a health-care provider or user. Anyone familiar with the arts understands that this closed- or semi-closed system could use any trans-mucosal extraction technologies and could be used for any blood chemical levels and medication pumps.

As to particular examples of receptacles useable for the system, these include, but are not limited to one or more functions of the transmucosal extraction devices receptacle is configured to serve, e.g., is selected from the group consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible trans-mucosal extraction patch, stretch stripes, adhesive stripes, trans-mucosal extraction patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a trans-mucosal extraction gauge, trans-mucosal extraction dipstick, trans-mucosal extraction rod, trans-mucosal extraction stick, biteplate, bendable or flexible trans-mucosal extraction device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent trans-mucosal extraction and electronic for analytical and other functions and applications in order to provide accurate physiological information, smart device to isolate, sports and recreational performance, animal sports and recreational performance, and other medical diagnostics, and analytics function, etc. with biosensors and transmucosal extraction devices and an RFID, micro- and nano-sensors and transmucosal extraction devices, micro- and nano-electronics, micro- and nano-enabled energy harvesting, micro- and nano-energy storage, micro- and nano-devices micro- and nano-programmable processors, micro- and nano-memory devices, micro- and nano-integrated power management devices, micro- and nano-programmable hardware, micro- and nano-wireless devices with communication capabilities across multiple frequencies located in an oral cavity or integrated outside of the oral cavity, and a device configured to be inserted into an animal or human's oral cavity, but not swallowed, one or more sensors and transmucosal extraction devices contained within or upon the receptacle, and at least one interface with a network capable of utilizing the information obtained from the one or more sensors and transmucosal extraction devices, one or more platforms, or one or more auxiliary devices or body integrations.

The smart sensors and transmucosal extraction devices receptacle could be configured with WiFi connectivity and is configured to provide an alerting signal when outside a pre-set range; the smart sensors, full server access and is configured for an analytical processing capability comprising racing performance analysis.

The network units of the system include ones capable of utilizing the information obtained from the one or more sensors and transmucosal extraction devices and having functions including, but not limited to, data storage, data retrieval, data synthesis, alert programs, data management, characterization, filtering, transformation, sorting, processing, modeling, mining, inspecting, investigation, retrieval, integrating, dissemination, qualitative, quantitative, normalizing, clustering, correlations, computer derived values and ranges, simple or complex mathematical calculations and algorithms, statistical, predictive, integrative, interpretative, exploratory, abnormality seeking, data producing, analyzing historical or previous data from same or different individual or team, visualizing or presenting development platforms.

While there is a focus on the sensors and transmucosal extraction devices insertable and securely attachable in the oral cavity, the system could, and in many embodiments, does include at least one auxiliary sensors and transmucosal extraction device receptacle selected from, but not necessarily limited to, the group consisting of one or more functions of the transmucosal extraction devices receptacle is configured to serve, e.g., is selected from the group consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible trans-mucosal extraction patch, stretch stripes, adhesive stripes, trans-mucosal extraction patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a trans-mucosal extraction gauge, trans-mucosal extraction dipstick, trans-mucosal extraction rod, trans-mucosal extraction stick, biteplate, bendable or flexible trans-mucosal extraction device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent trans-mucosal extraction and electronic for analytical and other functions and applications in order to provide accurate physiological information, smart device to isolate, sports and recreational performance, animal sports and recreational performance, and other medical diagnostics, and analytics function, etc. with biosensors, transmucosal extraction devices, sensors, cameras, audio speakers, an RFID inside or outside of a mouth, micro- and nano-electronics, micro- and nano-enabled energy harvesting, micro- and nano-energy storage, micro- and nano-device, micro- and nano-timer, micro- and nano-programmable processors, micro- and nano-memory, micro- and nano-integrated power management, micro- and nano-programmable hardware, micro- and nano-wireless communication capabilities across multiple frequencies, sensors and transmucosal extraction devices located in a mouth or integrated outside of a mouth, and an animal toy which is configured to be sucked but not swallowed.

The system includes one or more network units which could be configured to carry out a functionality including or consisting of signaling bi-directional transmissions to a secure server through one or more of WiFi, Bluetooth, GPS, and NFC, temporarily storing information in the smart device, and bi-directionally transmitting alerts to pre-selected devices or pre-selected personnel. Further network units employable in the system include or consist of, one or more RFID components, one or more cloud applications, a real-time or near-time slumber to alert mode, a manual control diagnosis mode, a programmed automated diagnosis mode, a geographic analysis mode, a species classification analysis mode, a disease specific or situational alerting mode, a function by which the one or more oral sensors and transmucosal extraction devices is activated and inactivated by another sensors and transmucosal extraction device, device or remote controller, and transmission through WiFi or other wireless modes.

In additional embodiments, the system includes at least one auxiliary smart sensors and transmucosal extraction device receptacle not configured to be inserted in an oral cavity of an animal or human. The system can include a network configured to analyze one or more performance parameters of a team sport or group activity.

The system including the auxiliary smart sensors and transmucosal extraction device receptacle can also include a network including one or more RFID components, micro- and nano-electronics, micro- and nano-enabled energy harvesting, micro- and nano-energy storage, micro- and nano-devices, micro- and nano-electronics, micro- and nano-enabled energy harvesting, micro- and nano-energy storage, micro- and nano-devices, micro- and nano-timer, micro- and nano-devices, micro- and nano-programmable processors, micro- and nano-memory, micro- and nano-integrated power management, micro- and nano-programmable hardware, micro- and nano-wireless communication capabilities across multiple frequencies located in a mouth or integrated outside of a mouth, one or more cloud applications, a real-time or near-time slumber to alert mode, a manual control diagnosis mode a programmed automated diagnosis mode, a geographic analysis mode, a species classification analysis mode, a disease specific or situational alerting mode.

The system can include one or more medical devices or medication dispensers. Further enhancements include a fully integrated treatment facility, a system in which one or more sensors and transmucosal extraction devices is activated by another sensors and transmucosal extraction device, device or remote controller, network modes including transmission through WiFi or other wireless modes, and systems in which at least one auxiliary smart sensors and transmucosal extraction device receptacle for a sensors and transmucosal extraction device is configured to serve one or more secondary functions within the animal or human's oral cavity.

In a further aspect of the invention, applicants have provided a device configured to be inserted and securely attached in an oral cavity of an animal or human. The device includes a smart sensor and transmucosal extraction device receptacle for sensors and transmucosal extraction device, and the receptacle is configured to serve one or more functions within the animal or human's oral cavity without being swallowed. The device also includes one or more sensors and transmucosal extraction devices contained within or upon the receptacle.

The one or more functions served by the receptacle of the device could include or consist of functions selected from the group consisting of, but not limited to, replacing missing teeth or parts of teeth, repairing broken teeth, providing an aligning, fixing of malpositioned teeth or jaws, or other corrective function, providing a cosmetic or cleansing function, assisting in proper breathing, eating or swallowing, providing a tongue thrust dental guard function, providing a pacifying function, providing a physiological, natural or grinding protective function, and providing a recreational or sports function, health analytics, diagnostic analytics, performance analytics; integration of body sensors and transmucosal extraction devices, health-devices, nano-particles, and sports and performance sensors and transmucosal extraction devices on inanimate objects and sports equipment; customizable developers' tool kit for biosensors, sensors and transmucosal extraction devices, performance, medical analytics, oral and systemic body diagnosis; integrated, pre-integrated and post-integrated, platforms; any type of medium, secure bidirectional media, multiple media, video, audio, 3D, printing, reporting, analytics, reporting, metadata diagnosis, with geometric tracking, communication networks, analytics, alerting, kinematics for individuals, team sports, organizational groups, animals and humans, communications, software management, data management, instant and long term animal and human analyses, multimedia inputs, visualizations, geometric motion, tracking, kinematics, alerting, therapeutic, electronic medical records, historical analysis, time stamped data, reporting and feedback, positioning, the integrated video can be synced with all wearables and other biosensors and transmucosal extraction devices in order to produce computer-generated precise movement and greater precision and analytics.

In certain embodiments, the above device serves at least to provide one of the following functions: replacing missing teeth or parts of teeth, repairing broken teeth, providing an aligning, fixing of malpositioned teeth or jaws, or other corrective function, providing a cosmetic or cleansing function, assisting in proper breathing, eating or swallowing, providing a tongue thrust dental guard function, providing a pacifying function for infants, children, elderly, and animals, providing sleep-aid and preventing snoring and obstructive sleep apnea function, therapeutic, providing a recreational, athletic performance, or sports function, physiological, natural and/or grinding protective function, horse-rider navigation and communication function, vital sign measuring function, diagnostic and therapeutic function, and surgical equipment and hospitalization equipment function.

Examples of particular devices embodying the invention include wherein at least one of the functions includes one or more functions of the transmucosal extraction devices receptacle is configured to serve, e.g., is selected from the group consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible trans-mucosal extraction patch, stretch stripes, adhesive stripes, trans-mucosal extraction patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a trans-mucosal extraction gauge, trans-mucosal extraction dipstick, trans-mucosal extraction rod, trans-mucosal extraction stick, biteplate, bendable or flexible trans-mucosal extraction device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent trans-mucosal extraction and electronic for analytical and other functions and applications in order to provide accurate physiological information, and any combination thereof.

The device receptacle is insertable, e.g., by micro- and nano-clips, frames, brackets, sealants, dental composites, bonds, adhesives, adhesive strips, cements, wires, bands, glues, embedment, injection, printing, tattooing, or any combination thereof. The device as herein described can be securely attached within the oral cavity of an animal or human by a number of means, including one or more of being fixedly inserted, imbedded, fitted, fixed, implanted, fastened, joined, associated, coupled, linked, banded, united, mounted, combined, glued, adhered, cemented, or firmly connected by mouth, e.g., lips, teeth, etc., or hands or parts or accessories thereto of either.

In these devices, the receptacle can be configured to be removable, self-removable, self-installable, coverable with an air-tight material, and configured so that the one or more sensors and transmucosal extraction devices is integral to the receptacle.

The sensors and transmucosal extraction devices in the provided devices can include sensors which measure a blood, interstitial fluid, lymph node, bone, or tooth constituent, sensors and transmucosal extraction devices which measure a predetermined biologic, sensors which measure a predetermined biologic including or consisting of DNA, RNA, telomeres, methylated or otherwise modified DNA or RNA, proteins, immunoglobins, antibodies, histones, peptides, modified proteins, neuro-peptides, pigments, and enzymes, sensors which measure dissolved gases, including oxygen, carbon dioxide, carbon monoxide, ammonia, sulphur, or an alcohol-containing gas, sensors which measure a lipids profile, sensors which measure a chemical molecule, sensors which measure a salt, an alcohol, a metabolite, an anion, a cation, water, a sugar, a protein, or a lectin, sensors which measure a drug or a medication, sensors which measure cells, the one or more sensors measures cells, cancerous cells, biomarkers for an oral or systemic infectious disease, biomarkers for drug abuse, biomarkers for a metabolic disease, biomarkers for malnutrition, biomarkers for obesity, biomarkers for a cardiovascular disease, biomarkers for atherosclerotic, biomarkers for infection, biomarkers for auto-immune and other immune diseases, biomarkers for stroke, biomarkers for AIDs, biomarkers for multiple sclerosis, biomarkers for periodontal diseases, biomarkers for brain-function disorders, dementia, memory loss, depression, mental disease, Alzheimer's disease, mentally-challenged disorders, nervous system disorders, tracking or wandering, and other psychology and neurological disorders, biomarkers for bleeding, head and neck injuries, biomarkers for Sjogen's syndrome, biomarkers for oxidative stress, biomarkers for allergies, biomarkers for cancer, biomarkers for skeletal and muscle diseases, biomarkers for genetic diseases, biomarkers for renal diseases, biomarkers for osteoporosis, biomarkers for fatigue, biomarkers for stress, biomarkers for sleep deprivation or sleep apnea, biomarkers for fertility, pregnancy, ovulation, and reproductive system disorders, biomarkers for cystic fibrosis, biomarkers for respiratory or pulmonary diseases, biomarkers for diabetes and ketoacidosis, biomarkers for inflammation, biomarkers for age-related diseases, biomarkers for dehydration, biomarkers for halitosis, biomarkers for alcohol consumption, alcoholism or drug consumption or drug addiction, biomarkers for hypoxia, smoking-related diseases, toxins, or pollutants, biomarkers for poor-gait, biomarkers for Crohn's disease, biomarkers for dental caries, biomarkers for blood and circulatory disorders, biomarkers for ear, nose, and throat diseases, biomarkers for taste, Ageusia, Hypogeusia, or Dysgeusia, biomarkers for bad-breath related diseases biomarkers for chewing or mastication, biomarkers for digestive disorders, biomarkers for hepatic diseases, spleen, gall-bladder and pancreatic diseases, biomarkers for urinary system disorders, biomarkers for integumentary system diseases, biomarkers for endocrine, biomarkers for Cushing disease, biomarkers for Cystic Fibrosis, lymphatic, and excretory diseases, sensors which measure a cell surface component or a cellular marker or component, sensors which measure a pathogen or a microbe, sensors which measure administered foreign materials, medications, diagnostic molecules, drugs, biologically sensitive, derived, bio-mimics, or bioengineered molecules, sensors which measure an ingested molecule or its metabolite, including wherein ingested molecule is a pathogen, a microbial, an ingested toxin, an ingested allergen, an ingested food constituent, including a nutrient, a micronutrient, a fat molecule, a carbohydrate molecule, a sugar molecule, a protein molecule, or an amino acid, sensors which measure ingested medications, ingested foreign material, ingested drugs, an ingested diagnostic molecule, an ingested biologically sensitive molecule, an ingested nanoparticle, an ingested derived molecule, a biomimic, or an ingested bioengineered molecule, sensors which interact with at least one disease-related biomarker, such as, e.g., a disease-related biomarker related to a disease diagnosable by a sensors which includes or consists of sensors of blood pressure, core body temperature, heart rate, levels of a predetermined biologic, chemical or medication or their metabolites.

In a particular embodiment, the device can include an interface with at least one sensor and transmucosal extraction device or nano-particles not located within the oral cavity.

The systems and devices described as part of the OSWT-Transmucosal Extraction system as laid out in this application can be applied to obtain extensive sensors and transmucosal extraction device data and analysis, providing much needed information and assistance, as detailed herein. In particular, a method for obtaining sensors and transmucosal extraction device data from an animal or human is provided. The method includes the steps of locating or inserting a device configured to be inserted and securely attached in an oral cavity of an animal or human. The device includes smart sensors and transmucosal extraction device receptacle for sensors, the receptacle being configured to serve one or more functions within the animal or human's oral cavity without being swallowed. In the method, the smart receptacle is inserted already containing, or, alternatively, receives one or more sensors and transmucosal extraction devices after insertion, capable of providing or receiving information or analysis relevant to the animal or human. The method includes activating and/or monitoring the one or more sensors and transmucosal extraction devices, and transmitting or receiving at least some portion of the information or analysis to, from or among a network or networks capable of utilizing the information or analysis.

In various embodiments of the method, information can be transmitted securely to a plurality of remote devices monitoring the animal or human, or information can be transmitted securely to a plurality of remote devices monitoring a plurality of animals or humans.

The network capable of utilizing the information obtained from the one or more sensors and transmucosal extraction devices can include or consist of one or more network units having the function of data storage, data retrieval, data synthesis, alert programs, data management, characterization, filtering, transformation, sorting, processing, modeling, mining, inspecting, investigation, retrieval, integrating, dissemination, qualitative, quantitative, normalizing, clustering, correlations, computer derived values and ranges, simple or complex mathematical calculations and algorithms, statistical, predictive, integrative, interpretative, exploratory, abnormality seeking, data producing, comparative, historical or previous from same or different individual or team, visualizing or presentation development platforms.

The method can include network units which utilize preset ranges, dynamic preset ranges, or degrees of alerts from preset ranges for medical or performance analysis. Additionally, the described method can include network units which utilize biosensors and transmucosal extraction device or sensors measurements for pre-integration and post-integration analyses, as known by those skilled in such arts.

In another embodiment, a diagnostic or other system is provided which includes a device configured to be inserted and securely attached in an oral cavity of an animal or human, wherein the device includes smart sensors and transmucosal extraction device receptacle for sensors and transmucosal extraction device. The receptacle is configured to serve one or more functions within the animal or human's oral cavity without being swallowed, and the smart receptacle is configured to contain or receive one or more sensors and transmucosal extraction devices capable of providing or receiving information or analysis relevant to the animal or human. The system further comprises a smart auxiliary device which is wearable, attachable or insertable externally to the oral cavity. The auxiliary device is configured to obtain information from, provide information to, or both, the one or more sensors and transmucosal extraction devices contained in, on, or received by the oral cavity receptacle. The one or more oral cavity sensors and transmucosal extraction devices or the auxiliary device, or both, are configured to transmit or receive the information or analysis to or from a network or networks. This system can include an auxiliary device which is also configured to contain or receive one or more sensors and transmucosal extraction devices.

The one or more functions can include or be selected from the group consisting of replacing missing teeth or parts of teeth, repairing broken teeth, providing an aligning, fixing-malpositioned teeth or jaws, or other corrective function, providing a cosmetic or cleansing function, assisting in proper breathing, eating or swallowing, providing a tongue thrust dental guard function, providing a pacifying function, providing a recreational or sports function, providing a physiological, natural or grinding protective function, or providing a function related to health analytics, diagnostic analytics, performance analytics; integration of body sensors and transmucosal extraction devices, health-devices, nano-particles, and sports and performance sensors and transmucosal extraction devices on inanimate objects and sports equipment; customizable developers' tool kit for biosensors and transmucosal extraction devices, sensors, performance, medical analytics, oral and systemic body diagnosis; integrated, pre-integrated and post-integrated, platforms; any type of medium, secure bidirectional media, multiple media, video, audio, 3D, printing, reporting, analytics, reporting, metadata diagnosis, with geometric tracking, communication networks, analytics, alerting, kinematics for individuals, team sports, organizational groups, animals and humans, communications, software management, data management, instant and long term animal and human analyses, multimedia inputs, visualizations, geometric motion, tracking, kinematics, alerting, therapeutic, electronic medical records, historical analysis, time stamped data, reporting and feedback, positioning, the integrated video can be synced with all wearables and other biosensors and transmucosal extraction devices in order to produce computer-generated precise movement or greater precision and analytics.

In certain embodiments of this system, the animal is a pet, including a dog or cat, or other animals. In embodiments of this system involving pets, the auxiliary device can be a smart collar. Moreover, in systems involving pets, in specific embodiments, the system can include a database compilation of one or more attributes of animals having at least one similar characteristic to the pet and which connects via WiFi to an access portal and wherein the system provides diagnostic information regarding the pet. Similarly, for pets, the system can include a database compilation of one or more of the pet's biological or physiological attributes.

Generally, the system can include a historical database of the animal or human as to one or more characteristics from which comparisons or analyses are configured to be made, or a database of animals or humans having a common characteristic to the animal or human on which the smart device is located and for which a predetermined comparison is configured to be made.

In another embodiment, the auxiliary device comprises a smart device configured to be associated with one or more team members of a team wherein the auxiliary smart device comprises one or more sensors and transmucosal extraction devices configured to obtain information from the one or more team members and is configured to transmit the information or analysis derived therefrom directly or indirectly to a network.

In one embodiment of this system, the auxiliary oral cavity device can be a mouth guard and the one or more auxiliary device sensors and transmucosal extraction devices can measure, e.g., oxygen levels or heart rate, analyze fatigue, such as the fatigue of an individual team member, or the fatigue of a composite of a plurality of team members. The system can also be configured to analyze kinematics, such as where the network is configured to analyze the kinematics of an individual member of a team or group or a composite of a plurality of team or group members.

The system can be set up for use with an individual to obtain information from the individual and transmit it or analysis derived from it directly or indirectly to a network.

The system network can interface with a mobile device which in turn provides sensors and transmucosal extraction device information or analysis to the individual user, who then receives information feedback regarding a physiological characteristic of a current activity he is engaged in, such as running, jogging, walking, sleeping, or a physical characteristic involved with playing a sport.

The system may utilize a network configured to analyze one or more performance parameters of a team sport or group activity. It may also work with a network configured to analyze one or more performance parameters of horse racing. Additionally, in an additional embodiment, the system can include a network configured to provide electronic medical records functionality.

The system as described above can include full server access, and the system can be configured to analyze individual or team sports performance as it relates to various body components and sensors and transmucosal extraction devices.

In one option of the system, the system includes one or more of a digital storage device or full connectivity capability configured to analyze individual and team sports performance as it relates to various body component sensors and transmucosal extraction devices for post-play analysis and review.

The tool kit or platform can also include the components and, optionally instructions, e.g., to provide a system with an electronic medical records functionality.

In yet another aspect, one or more sensors and transmucosal extraction devices are designed to be insertable within or upon the pacifier and constructed to be interchangeable or securely affixed to the pacifier. The system in which the sensors and transmucosal extraction devices is insertable and is configured to be securely affixed to the pacifier may also be configured to be exposed to oral tissues.

The method for monitoring blood components can include data storage, e.g., through a digital storage device, connector, or other medium integral to or separate from the blood monitoring device.

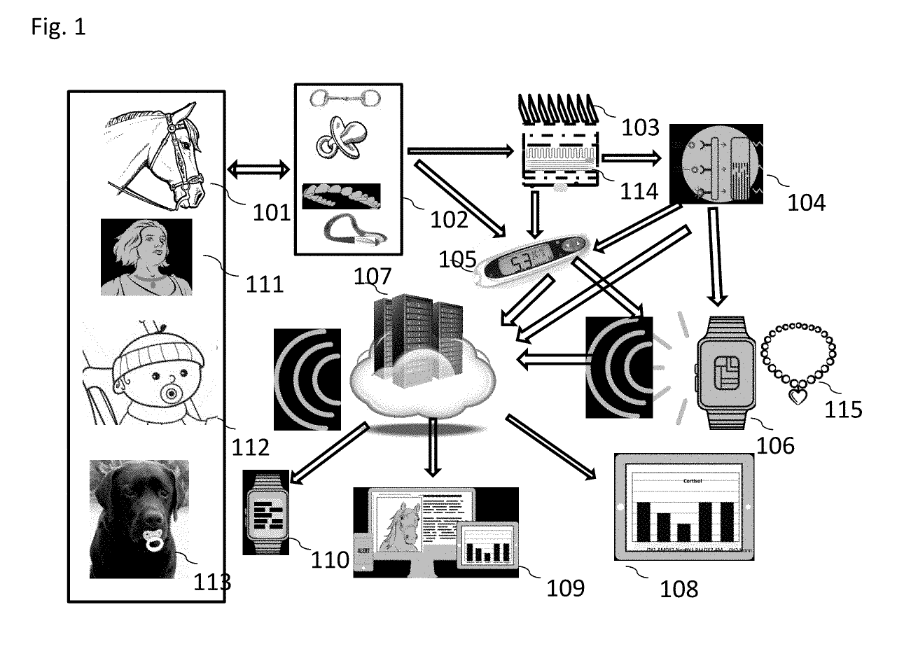

FIG. 1 is a schematic depiction which exemplifies an OSWT-transmucosal extraction and transmucosal extraction device system secure wireless communication capability and information retrieval through the animal's or human's oral cavity according to the invention. In these embodiments of the described OSWT-transmucosal extraction or transmucosal extraction device, a human, here exemplified by a woman 111, can have OSWT-transmucosal extraction or transmucosal extraction device attached to her teeth through a retainer, or another dental devices such as infant or animal pacifier, equine-bit or equine cheek or lip clip, 101, 111, 112, 113. Biologics or biologically relevant molecule including, but not limited to, blood sugar or glucose, hydration levels, or electrolyte would be extracted by trans-mucosal from blood and interstitial fluid and then either detected by sensor in retainer, infant or animal pacifier, equine-bit or equine cheek or lip clip 102 or by reader 105; blood components could be processed prior to detection and analysis by the reader. Not shown here but in some cases, the extracted blood components could be shipped to a laboratory for further processing, detection, and analysis by a reader. Alternately, gathered information can be transmitted via the Internet of things where internet communication chips with their own IP address and provides the ability to transfer data over a network contained in the OSWT-transmucosal extraction in oral cavity device 103, 104 or in the reader 105. In some embodiments, information gathered could be stored in OSWT-transmucosal extraction 104, 104 or the reader 105. The smartwatch, necklace, other accessories, or wearables 106, 115 transmits the oral cavity information to one or more remote PC laptops, tablets, smart-phones, wearable, or reader communication devices through the owner's WiFi in accordance with embodiments of the present invention 108, 109, 110. Sensors device could be inserted in the oral cavity such that the sensors bath in the extracted blood components to measure blood glucose levels, blood composition, medication, etc. 104. In one embodiment of the arts, the blood components could be concentrated and isolated by a chip similar to lab-on-a-chip before exposure to sensors 114. As needed, the information or signal can then be transduced, amplified, and processed 104. The resulting signal can be transmitted, e.g., through a RFID tag or blue tooth 5, to a RFID reader or a piconet for blue tooth network on an accessory, smart jewelry, clothing, watch, other accessories, on, in, or around the woman, exemplified here by a smartwatch 106 or smart necklace 115. The OSWT-trans-mucosal extraction system can include an RFID tag reader or blue tooth chip placed within or in proximity to any part of the oral cavity, temporarily or permanently. The signal is then transmitted to a secure server or cloud 107. Information could be stored or sent from the server or cloud to several different smartphones, smart watches, computers, and others. The alert can be dispatched to any computer-aided device or emergency dispatch if the OSWT-transmucosal extraction device or transmucosal extraction reader system detects higher than average or abnormal metabolic ranges, for example.

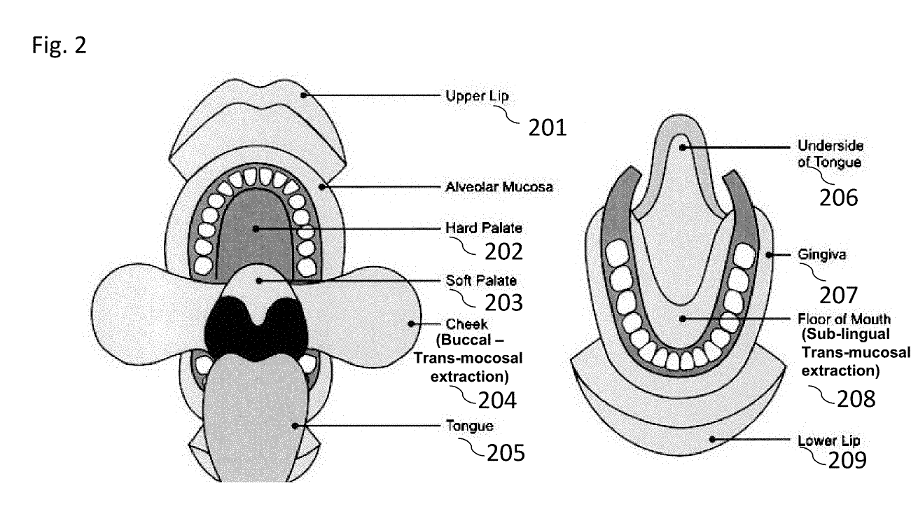

FIG. 2 is a schematic depiction showing which oral cavity tissue or portions could be most useful for diagnostic measurements. The oral cavity consists of the upper and lower lips 201 and 208, cheek 204, tongue 205, hard palate 202, soft palate 203, gingiva 206, and floor of the mouth 207. The lining of the oral cavity is called the oral mucosa and includes the buccal, sublingual, gingival, palatal and labial mucosa. Because cheek (buccal) mucosa 204 and through the floor of the mouth (sublingual) mucosa 207 have non-keratinized cells and form lesser of the barrier, they are better suited for transdermal extraction for blood or, interstitial fluid chemistry analysis. Other parts of the oral cavity can also be used for transdermal extraction, but due to the presence of keratinized cells, they might be less efficient for some techniques. Still, due to the abundant blood supply, oral mucosa is better suited for transmucosal extraction than the skin.

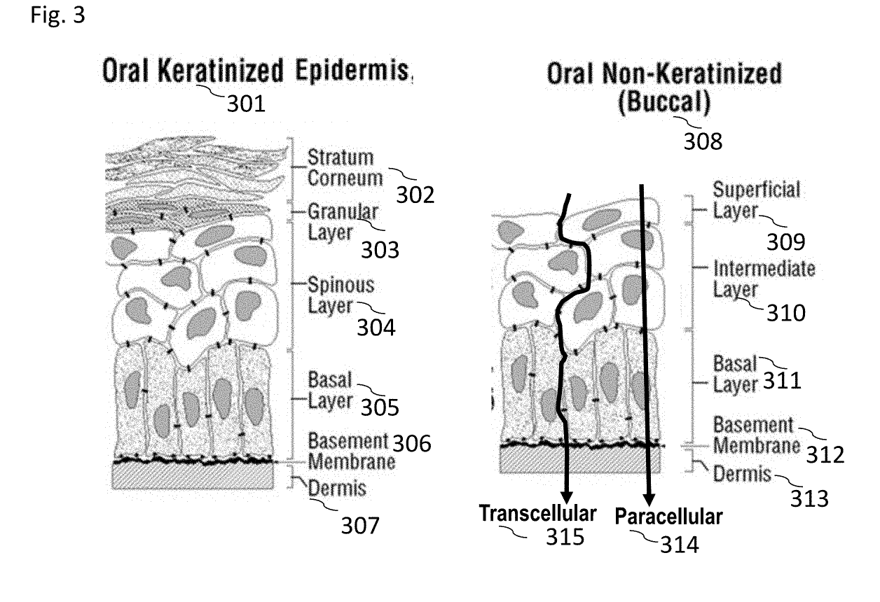

FIG. 3 is a depiction of the cellular structure of oral mucosa: The oral mucosa is of two types, keratinized 301 and non-keratinized 307. The mucosa of areas that are subjected to mechanical stress such as the gingiva and hard palate is keratinized and is similar to the epidermis of the skin 301. However, the mucosa of the soft palate, sublingual, and buccal regions are not keratinized and form less barrier for transmucosal extraction 307. The keratinized epithelia contain neutral lipids like ceramides and acylceramides are associated with the barrier function 302. These epithelia are relatively impermeable to water. In contrast, non-keratinized epithelia, such as the floor of the mouth and the buccal epithelia do not contain acylceramides and only have small amounts of ceramides. They also contain small amounts of neutral but polar lipids, mainly cholesterol sulfate and glucosyl ceramides. These epithelia have been found to be considerably more permeable to water than keratinized epithelia 308. Beneath the epithelium is the basement membrane, lamina propia and submucosa 304, 305, 309, and 310. The oral mucosa also contains many sensory receptors including the taste receptors on the tongue. Buccal or lingual extraction can be via passive diffusion across lipid membranes via either the paracellular 313 or transcellular 312 pathways. Both blood and interstitial fluids components can be easily analyzed via transmucosal extractions.

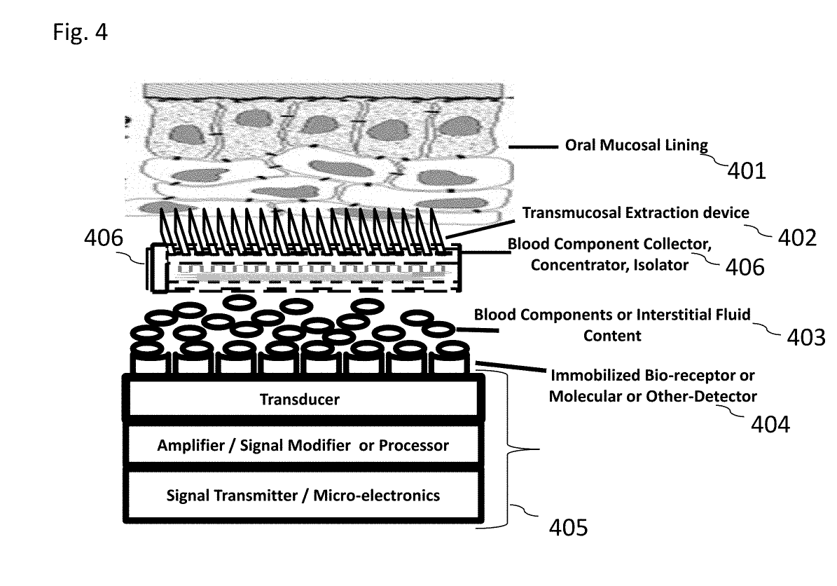

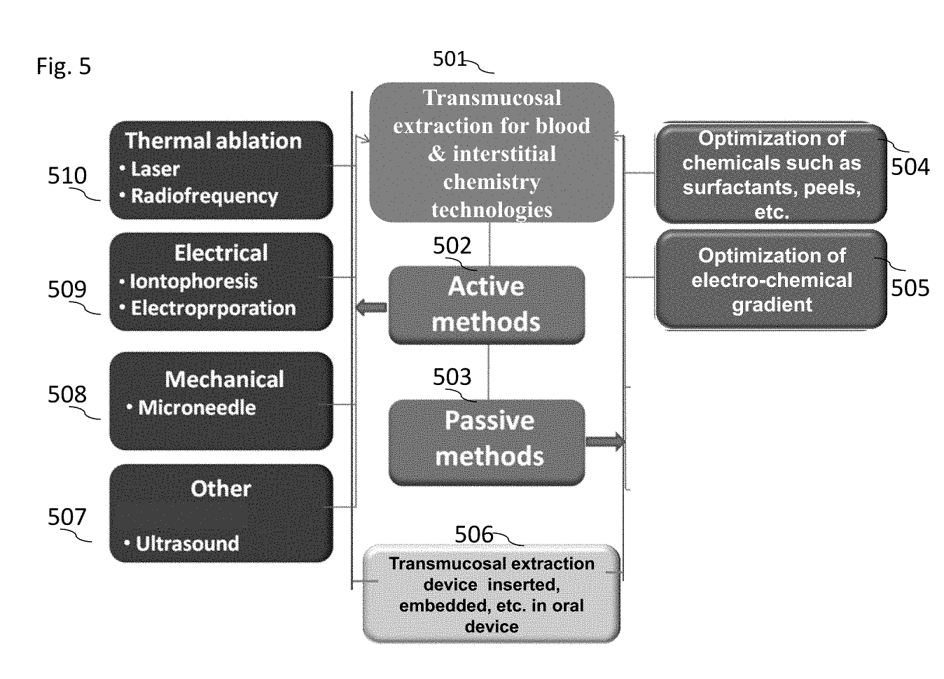

FIG. 4 exemplifies the structure of a transmucosal extraction device along with the sensors or biosensors, forming OSWT-transmucosal extraction system, as may be used in embodiments of the invention. The transmucosal device could be in contact with mucosal lining 401 and 402. The transmucosal extraction device could utilize one or more passive or active technologies described in detail in FIG. 5. The biologically active substances, e.g., blood biomarkers, blood, or interstitial fluid chemicals, or biologically sensitive materials are exposed to biosensors such as bio-detectors, bio-receptors, etc. 403 and 404. Bio-detectors, bio-receptors, or biologically sensitive materials 404 include, but are not limited to, biologically-derived materials, bio-mimics, chemicals, bioengineered molecules, ligand, or ligand-binding molecules, optical sensors, etc. The sensors interact with blood components, interstitial fluid molecules, biological molecules, drug molecules, biomarkers, etc. and this recognition is converted by transducers 405 to a signal that is more readily measured and quantified. A transduced signal is transferred to a signal amplifier or modifier, which transfers the signal to a wireless transmitter in this embodiment. The transmucosal extraction device could contain software for the device functionality, one or more electrical input; one or more transducers configured to convert input electrical energy to different energy forms for activating and implementing extraction procedure; a controller configured for providing the control signals, intensity, duration, timing, sequence, and nature information for the different energies supplied to the device said transducers; and one or more compartment configured for either collection of extracted fluids and/or detecting the blood and interstitial components 406. It is understood by anyone familiar with the arts that the entry and control of entry of blood components could be controlled by use of a micro-pump or a microinfusion pump. It is understood by anyone familiar with the arts that the organization modules or device parts such as transmucosal extraction device, a software module, transducers, controller, collection and concentration chips, etc. presented here could be organized in a variety of manners depending upon ease of use, manufacturing feasibility, and regulations. It is understood by anyone familiar with the arts that this device or the reader could contain preservatives, anticoagulation, coagulation enhancers, and other chemicals routinely used to enhance detection and storage of collected fluids. Furthermore, the device could also contain or be coated with pain-killers or pain-numbing agents for patient comfort. These devices could be custom-made by 3D printing.

FIG. 5 illustrates various technologies to enhance diagnosis through transmucosal extraction: The transmucosal extraction can be enhanced either by passive or by active methodologies 502 and 503. Passive methods require no energy or physical mucosa disruption for mucosa permeabilization and extraction and could include optimization of chemicals such as surfactants, peels, optimization of electro-chemical gradient 504, 505, 506. Our device can also be used with active methods, which require either energy or physical mucosa disruption for mucosal permeabilization and extraction, and could include ultrasound, electrically assisted methods (electroporation and iontophoresis), thermal approaches (lasers and radio-frequency heating) and mechanical methodologies such as microneedles (MN) and tape stripping, and ultrasound 510, 509, 508, and 507. These active, passive, or a combination of both technologies based trasnmucosal extraction devices could be embedded, inserted, permanently or temporarily attached by several methods detailed elsewhere into oral mucosa 506. These approaches allow for an accurate and consistent detection and analysis of blood and interstitial fluids components and others to be detected and analyzed. Chemical and electro-chemical gradient based are salt, natural, man-made polymers and compound assisted diffusion, inhibitors, surfactants, enzymes, salts, chemical- or enzyme-peels, liposomes, enzyme inhibitors, nanoparticles, polymer-chain, microparticulate, mucoadhesives, diffusion, osmosis, etc. 504 and 505. Active methods involve the use of external energy to act as a driving force for transmucosal extraction across the mucosa or by physically disrupting the keratinized or non-keratinized mucosa. It is understood by anyone familiar with the arts that these technologies could be used for drug delivery via trans-mucosal similar to trans-mucosal extraction. It is understood by anyone familiar trans-mucosal extraction device or a part thereof could be flexible, non-flexible, moldable, contain micro- or nano-hinges, -pivots, -hinges with micro- or nano-teeth, dial, others, or a combination thereof. This design could ensure proper and efficient contact between the device and the oral tissue. Furthermore, the contact between some or all parts of the device could be customized via flexible, in-flexible, mouldable, micro- or nano-hinges, -pivots, -hinges with micro- or nano-teeth, dial, others, or a combination thereof to each individual for best drug delivery. These could also assist in the manual or automatic control of most efficient the oral tissue contact area, contact angle, contact time, etc.

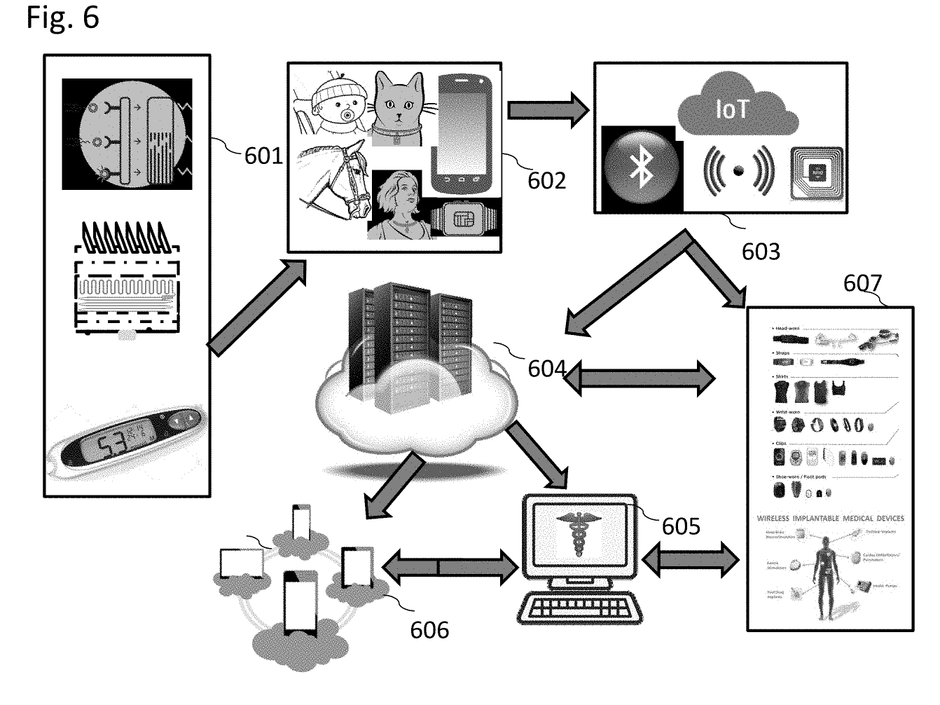

FIG. 6 depicts embodiments which exemplify OSWT-transmucosal extraction dynamic alerting software and secure networks, in accordance with embodiments of the present invention. In these embodiments of the described OSWT-transmucosal extraction system an example of a fully integrated diagnostic and performance measurement system is provided. 604 represent a secure host server or cloud networks which includes all forms of smart devices, one or more pagers, SMS, Faxes, emails, GIS mappers, beacons (XYZ) telephones, PSTN devices (Voicemail, IVR, ASR, TTS), satellite phones, wireless communications systems, and other forms of communication which can be implemented and utilized by one or more individuals, one or more animals, or one or more organizations 605, 606. In addition, the present invention can include a privatized internal server host and subsystems as well as one or more external hosted alert servers. A plurality of collective data can be derived from several OSWT-transmucosal extraction system measurements including, but not limited to, the integration of any type of wearable, sensors, and medical devices such as implantable medical devices, on body sensors and wearable and environmental sensors and devices 607 and other embodiments in the present invention. A plurality of sensor and biosensor data from OSWT-transmucosal extraction devices or transmucosal extraction devices and reader could inform all smartwatch, pacifier, animal collar, necklace, and other smart devices through a variety of wireless technologies such as Wi-Fi, Bluetooth, Internet of Things, MiFi 601, 602, 603. All wearable devices whether smart or not smart, all RFID readers, all can be examined and analyzed in order to determine the degree of an alert (low, medium or high) being dispatched through various templates referred to as friends and family which includes trainers, caretakers, and other authorized personnel, and electronic medical records 605, 606

The oral cavity is a semi-sterile, clean, fast wound healing environment, and has a high threshold for pain because oral secretions include antiseptic-like molecules, wound healing, and pain-killer biologics which are unique to this cavity. In animals, these properties are apparent when they groom themselves by using their tongue to lick away dirt and pathogens, yet no infection results from the licking of pathogen-laden dirt.

Oral mucosa is highly vascularized and the blood supply to the mouth represents systemic blood supply because, unlike stomach, nutrient absorption is not the primary function of the oral mucosa. This ample blood supply could assist in collecting enough blood components via transmucosal extraction, which is often not possible for transdermal extraction devices. The Academy of General Dentistry suggests that more than 90 percent of all systemic diseases, diseases that affect or pertain to the entire body and not just one of its parts, produce oral symptoms and are reflected in oral secretions. Thus, the oral cavity serves as a critical vantage point for detecting the early onset, signs, and symptoms of diseases including, but not limited to, systemic infections like AIDS, cardiovascular diseases, atherosclerotic inflammation, and stroke, preterm delivery with low-birth weight babies, dental diseases, and tooth decay. It is a site for oral cavity infections that damage teeth and gums.

Oral cavity also has several characteristics that make it a great place for blood chemistry analysis via both blood and interstitial fluid analysis and biological detection. Mucosa of oral cavity has abundant blood supply, is fast wound healing without scaring, and contains areas of high solvent permeability. This high permeability is related to the oral tissue and mucosa cellular structure. Based on morphology, structure, and differentiation oral mucosa is of three different types, masticatory mucosa which is keratinized stratified squamous epithelium, lining mucosa which is non-keratinized stratified squamous epithelium and specialized mucosa which contain taste buds. The gingiva and hard palate are like skin tissue are stratified and keratinized squamous epithelia and is usually tightly attached to underlying structures by a collagenous connective tissue. A critical characterized of stratified, keratinizing epithelia is that the cells undergo a terminal differentiation that results in the formation of a tough and mechanically resistant surface composed of cornified cells. These cells are filled with keratin filaments and lipids, and they lack both nuclei and cytoplasmic organelles and the cell membrane is replaced by a proteinaceous cornified envelope that is covalently cross-linked to the keratin filaments. These features make mastication mucosa into a highly insoluble yet flexible structure that protects the underlying epithelial cells. This mucosa like skin is a strenuous barrier that restricts passive diffusion to small (<500 Da) lipophilic and hydrophilic molecules.

The dorsal surface of the tongue contains specialized epithelia with taste buds with nerve endings for general sensory reception and taste perception. The dorsum of the tongue has a surface consisting of areas of both keratinized and non-keratinized epithelium; these are tightly bound to the underlying muscle of the tongue.

Buccal and sublingual oral lining mucosa epithelia is like esophagus, uterine, and cervix epithelia in that they are all made of a non-keratinized stratified squamous epithelium and can vary considerably in thickness in different oral regions. The lack of keratinized, stratified the epithelia in the buccal and sublingual surfaces enhances solute and solvent permeability. The keratinized mucosa represents approximately 25%, the specialized mucosa (dorsum of tongue) approximately 15%, and the non-keratinized mucosa approximately 60% of the total oral surface.

Several techniques are used to test and analyze blood and interstitial fluids for diagnostic screen through skin are iontophoresis, electroporation, reverse iontophoresis, ultrasound, sonophororesis, phonophoresis, electro-gradient based, laser, radio frequency heating, micro-needle, tape stripping, etc. Such measurements of blood and interstitial fluid components for diagnostic purposes are commonly referred to as “blood chemistry.”

Iontophoresis and electroporation are two major means of electrically-facilitated transdermal extraction systems. During electroporation, cells are temporarily exposed to high intensities of electric pulses that cause formation of aqueous pores in the lipid bilayers of the skin, so the blood and interstitial fluid components can diffuse across skin. Unlike other transdermal enhancement methodologies, it acts mainly by involving a second driving force, the electrical potential gradient as companion to the concentration gradient across the skin since uncharged species can also be delivered by electro-osmosis. However, the main side effect of these cellular pores is the resultant cellular injury and even cellular death.

Thermal ablation is anther technique used for transdermal extraction and is of two types, laser and radio frequency. The mechanism of laser thermal ablation of the skin is the selective removal of the top layers without damaging deeper tissues, so enhancing the extraction of hydrophilic and macromolecules molecules into the sensing device. Lasers ablate the superficial layers by use of optical energy and formation of micro-channels in the skin. Radiofrequency (RF) thermal ablation is performed by the placement of a thin, needle-like electrode directly into the skin and application of high frequency alternating current which produces microscopic pores in the skin and through these pores blood and interstitial hydrophilic and macromolecules can be extracted.

Micro-needle array is another effective technique that can easily extract all types of molecules from blood and interstitial fluid by minimally invasive systems. Micro-needles are multiple microscopic projections typically assembled on one side of a supporting base. These micro-needles can overcome disadvantages commonly associated with hypodermic needle usage and increase and improve patient compliance. They are minimally painful, minimal possibility of infections, can be easily self-administration, and reduce instances of accidental needle-sticks injuries. These arrays can replace hypodermic and subcutaneous needle technologies, and they combine the ease of use of a transdermal patch with the effectiveness of conventional hypodermic needle and syringes. Pain can be easily minimized by creating needles of suitable length, width, shape, and placed to avoid nerve contact when inserted into the skin. They are usually designed in arrays in order to improve the surface contact with the skin and facilitate molecular extraction. They are designed to create transient aqueous conduits of micron dimensions across the skin. Since these micro-pores are orders of magnitude larger than macro-molecular blood components, they can readily permit the transport of all types of molecules in the blood and interstitial fluid.

Application of low-frequency ultrasound rapidly increased skin permeability for the transdermal extraction and is referred to as low-frequency sonophoresis. Low-frequency ultrasound can potentiate molecular extraction of wide range of blood compounds such as proteins, genes, hormones, lipids, drug particles, chemicals, enzymes, hydrophilic molecules, macromolecules, and others. Low-frequency sonophoresis offers advantages over other trandermal extraction methods because it can be controlled by varying ultrasound parameters. The size of aqueous skin pores thus created can be controlled by varying the frequency and the intensity of the ultrasound. Low-frequency ultrasound can be used alone or in combination with other methodologies.

These aforementioned commonalities are true for animals, including, but not limited to, pet, farm, zoo, wild, equine, and laboratory animals, as it is true for human and related animal diseases.

Unlike any other body cavities, the oral cavity represents a unique cavity because it is easily accessible, contains taste buds and lymph nodes, is highly vascularized and enervated. In addition, salivary secretions also contain fast healing and analgesic properties. These properties are experienced by most people during their daily teeth cleaning routine that often causes painless and fast healing gum bleeding. This example demonstrates the naturally-occurring aseptic, pathogen-destroying, and natural healing properties of saliva. Compared to currently used body parts, such as skin (which in animals adds extra complications due to the presence of fur) used to place or implant sensors and transdermal extraction devices, oral tissues are less painful, heal fast, and are more comfortable since any broken tissue during sensors and transmucosal extraction placement or insertion is fast healing, has less of a possibility of infection, and in most cases, would involve a one-time wound. These qualities make oral biosensors and transmucosal extraction and other sensors less invasive or minimally invasive. Discrete without being visible and yet easily accessible, they can be worn for a short time or round the clock, including during rest, sleep, high activity, and more. An increase in a patient, both human and animal, compliance would result from their non-invasive or minimally invasive nature and ease of accessibility. Thus, these transmucosal extraction devices, used in accordance with the invention, reduce healthcare cost while simultaneously increasing patient compliance. Oral biosensors, transmucosal extraction, and sensors used as described herein are superior to other biosensors and transdermal extraction devices in that they are able to measure biologics and biomarkers more accurately and consistently, intermittently, frequently, or constantly as needed to treat and manage most diseases. Once the biosensors and transmucosal extraction device are placed in the oral cavity, it can monitor and collect data for seconds, minutes, days, months, or even years. These biosensors and transmucosal extraction devices may be long-term or short-term, permanent or temporary. Non-oral biosensors and transdermal extraction that is attached to other body parts typically remain attached just a few hours.

Cheaper and personalized transmucosal extraction, biosensors, or sensors of the invention become important in managing healthcare costs and outcomes for multiple diseases. As technologies such as nano-technology, micro- or nano- electro-mechanical technologies, and/or 3D printing become more advanced and easily and cheaply available, the OSWT-transmucosal extraction or transmucosal extraction system of the invention may be adapted to provide additional savings in healthcare costs for both humans and animals. Furthermore, the present invention enables transmucosal extraction, biosensors, or sensors to be uniquely communicated from one or more animals and humans to a plurality of transmucosal extraction systems; thus, adding to the utility of the sensors and transmucosal extraction. The transmucosal extraction system of the invention enables humans to monitor and alert themselves or others, and enables animal-transmucosal extraction units to send alerts to humans in order to monitor and alert their caretakers. The present invention systematically integrates all forms of oral based transmucosal extraction, sensors, and biosensors in order to offer new, innovative, and unique opportunities to monitor the overall health status of both humans and animals. They also help to preemptively and accurately diagnose, detect, and monitor, and thus, help in the prevention, prognosis and risk-assessment of a variety of diseases. The oral cavity is a window into the overall health and disease status for the entire body and the transmucosal extraction system of the invention enables and integrates a multitude of the aforementioned devices to quickly and efficiently communicate a person or animal's heath landscape and schematic map. When blood drawn from a patient is sent to a laboratory for diagnosis, this involves an invasive, painful, costly, and time consuming process with frequent delays of usually days before diagnostic results become available to healthcare providers and patients. The present invention describes a needed precursor to, or substitution for, other more invasive methods of health and disease diagnoses. Patient non-compliance results from the painful blood draw which too often causes infections. Many chronic or frequent measurements simply would not need to be performed when substituted for with transmucosal extraction monitoring, which makes treatment more likely. Cost of healthcare increases enormously due to untimely and delayed diagnoses that interfere with proper treatment and good prognoses. Early and timely diagnoses for many diseases including, but not limited to, cancer, diabetes, cardiovascular diseases, and other metabolic diseases, could result in better prognoses, improved quality and quantity of life, and decreased healthcare costs.

The transmucosal extraction system of the invention is uniquely developed to provide highly customizable information and alerts. This is in part due to the variety of available sensors, and transmucosal extraction receptacles, accessory receptacles, network interfaces and network components that may be utilized and combined.

A feature of many embodiments of the OSWT-transmucosal extraction system and methods of the invention is the dynamic nature of the integrative alert components that may be built into the customizable operations. For example, people, individuals, groups, teams, hospitals, organizations, etc., may be alerted on a dynamic basis when one or more or variable combinations of, program range settings of various parameters are exceeded. The customizable alert modes may be operated as real-time or near-time slumber to alert, programmed, automatic, manual control, geographic-specific, disease or condition-specific, gender-specific, age-specific, species-specific, situation-specific, activation-triggered, or woken-up by another sensors and transmucosal extraction device, device or remote mode, or any combination of such customized parameters and components. The flow chart of an integrated OSWT-transmucosal extraction system includes one or more of movement, audio, video, pressure, impact, sensors and transmucosal extraction wearables, etc. which provide kinematics and other feedback to enhance accurate measurement of performance, movement, biometrics, and can add in health-related and physiological inputs as well to optimize health, well-being and/or performance criteria.

The OSWT transmucosal extraction system of the invention in one embodiment, for example, measures respiration rate, heart rate, blood pressure, temperature, blood O₂ levels, inflammation, etc. as a component of the “practiced” oral device insert. A “practiced” oral device insert may include any customized device inserted in an animal or humans' oral cavity and is designed to monitor any metabolic situation such as, e.g., metabolic diseases, cardiovascular diseases, cancer, infections, etc. OSWT-transmucosal extraction as used in the invention includes mobile device interfaces, which read a variety of diverse sensors, and transmucosal extraction devices. Moreover, with the discovery of new biomarkers, oral biosensors, and transmucosal extraction devices of applicant's invention are even more useful in detecting, diagnosing, risk-assessing, prognoses, and monitoring even more diseases. 3D printing applied to the invention makes cost-effective and less costly custom dental devices available and thus makes our OSWT-transmucosal extraction system more cost-effective. In one embodiment, the present invention involves pre-required diagnoses for treatment of a disease. Diagnostic testing is performed based on a patient's symptoms and history. Patient communication is one key element to gathering information about a patient's symptoms and history. Animals are unable to communicate their symptoms, so simple diagnostic tests such as frequent measurements of electrolytes, pH, respired gases, temperature, and blood pressure are utilized in determining their health and disease status. However, such diagnostic testing in many animals requires them to be sedated. Using one-time sedation to place or implant an OSWT-transmucosal extraction system of the invention, these oral transmucosal extractions, biosensors, and sensors offer a unique and novel opportunity to measure constantly or frequently as needed for these diagnostic purposes. The elderly, disabled, mentally ill, babies, and chronically ill could similarly benefit from frequent measurements. Conventional methods are limited by time and location of routine diagnoses like blood pH, electrolytes, temperature, blood pressure, or blood and interstitial fluids chemistry, etc. However, the transmucosal extraction, biosensors, and sensor system of the present invention offers a flexible and unique opportunity to measure these statistics at home, at work, during exercise, or even while sleeping. The device may be present in the oral cavity for a short (few seconds to minutes to few hours) or long time (several days to months to years), and may be temporary or permanent.

In yet another embodiment, the OSWT-transmucosal extraction systems of the invention may be used in conjunction with other smart wearables or attachables on one or more individuals creating a measurable team diagnosis. Furthermore, smart wearables or other medical devices may be utilized by a collective group, including in any team sports application, i.e., basketball, soccer, baseball, hockey, swimming, track, football, cricket, gymnastics, and other Olympic or global sports, etc. It is understood that the OSWT-transmucosal extraction system of the invention may measure and diagnose individual performances in sports (singularly measured, analyzed and diagnosed) or these may be measured, analyzed and diagnosed, collectively as a team composed of individual players. The device can be implanted, flexible, adhered, fixed, and/or cemented to the floor of the oral cavity, inside of the cheeks, tongue, and the sublingual cavity and/or affixed in other places within the oral cavity. The device locations within the oral cavity are optionally chosen by the patient or his/her caretaker and/or healthcare provider. The choice of location is one based upon a combination of factors including, but not limited to, convenience, comfort, durability, exposure duration to molecules to be measured, and duration of the device placement in the oral cavity and manufacturing and regulatory parameters. The transmucosal extraction, sensors, and biosensors may be placed by a healthcare provider including, but not limited to, physicians, surgeons, dentists, veterinarians, or healthcare provider assistants, caretakers, and/or patients themselves. The placement is usually made with the assistance of one of the following; for example, no sedation, mild sedation, full sedation, local anesthesia, general anesthesia, or other. Dental devices represent biologically inert surfaces; whereas, other areas of the oral cavity such as the gums or tongue, are biologically active and non-inert. The transmucosal extraction, sensors, and biosensors, therefore, may be placed fully on inert surfaces, active biological surfaces and/or a combination of both. The transmucosal extraction device would face oral tissue, whereas, sensors and biosensors may be exposed to elements of the oral cavity or covered with a barrier or a combination of both.

OSWT-transmucosal extraction materials inserted in the oral cavity as described in the present invention may detach, clip, be alerting chips, etc., and can be constructed for a short time period (temporarily) or constructed for a long-term (e.g., permanent) time period. All time periods for the plurality of orally inserted and attached devices used for one or more functions of the invention may be deployed. Oral devices employed include, but are not limited to, biosensors and transmucosal extraction devices, RFID tags, and any inserted attached or detachable dental device used for humans and animals to obtain data and sensors and transmucosal extraction-derived information. The design and construction of the inserted oral device will vary for each species and is customizable to detect, monitor and/or alert for all types of medical conditions.

In addition, in another embodiment of the present invention, the design of the OSWT-transmucosal extraction system is configured to help avoid the possibility of choking, for example; made of smooth (or partially or fully encapsulated or encased, oval, round, rounded or in any shape or size of similar utility) which could easily be swallowed and digested or passed through the digestive process; the biosensors and transmucosal extraction itself might not be smooth. OSWT-transmucosal extraction materials used herein are optionally made un-chewable or chewable depending on the species, medical need, and functionality and durability requirements. In addition, an alert can be sent when the sensors and transmucosal extraction or OSWT-transmucosal extraction device becomes semi and/or completely detached or travels to other parts of the body, such as the esophagus or stomach. The OSWT-transmucosal extraction material employed may be designed to dissolve or break apart in order to prevent choking which could be particularly useful in the case of animals, children, the disabled, and the elderly, and others who cannot communicate that the device was detached and swallowed.

In addition to oral transmucosal extraction devices and biosensors, the OSWT-transmucosal extraction system of the invention includes sensors, biosensors, drug delivery systems, transdermal extraction devices, and nano-particles located elsewhere within and/or on an animal or human that interfaces with the OSWT-transmucosal extraction system. The OSWT-transmucosal extraction system also can interface with various other devices located on or near an animal or human and securely communicates with a plurality of remote devices monitoring the health, disease status, therapeutic status, athletic performance, and/or well-being of one or more animals or humans. These devices are optionally within a human or animal body, e.g., such as, a drug dispensing pump or other. Alternatively, the device may be located on the human or animal's body, such as a cardiac monitor, dog collar, equine equipment, and accessories or other. These devices may read and interface with a variety of diverse transmucosal extraction devices, biosensors, and sensors such as blood component measurements of the OSWT-transmucosal extraction system with a cardiac monitor, a drug dispersing pump, muscular lactic acids sensor, sensors in sports-field as one example. The OSWT-transmucosal extraction system may also communicate with nano-particles in any body part.

In a further embodiment, one or more databases, secure servers, or other devices is utilized to store and/or capture data. The database contains data collected from the OSWT-transmucosal extraction system and any data input in the system including, but not limited to data used to send alerts; here, data encompasses both data captured and collected from OSWT-transmucosal extraction and data used to send out alerts. One or more software programs may collect, capture, and store data obtained from any of the OSWT-transmucosal extraction interfaces. Some of this data will be publicly available and viewable and some of the data will be only for private viewing and available to the relevant users and healthcare providers. To prevent any loss of data, in certain embodiments, when connectivity is lost and data cannot be transmitted in real time to a central storage system, data is temporarily stored on the local device and transmitted to a central system after connectivity is restored. These software programs may be responsible for any and all data related aspects such as, but not limited to, data comparative with historical or previous data of same individual or other, management, characterization, filtering, transformation, sorting, processing, modeling, data mining, queries, browsing, inspecting, investigation, retrieval, integrating, dissemination, qualitative, quantitative, symmetric, asymmetric, normalizing, clustering, correlations, computer derived values and rages, simple or complex mathematical calculations and algorithms, analytics, statistical data, predictive data, integrations, interpretation, exploratory, finding abnormalities, performance, data products, consumer data, server data, visualizing and/or presentation in a variety of platforms. Here, data analysis also means software for that analysis for disease and other diagnosis and analysis for both humans and animals. Thus, this software may supplement or partially and/or fully replace a healthcare provider's input, such as that of a physician, veterinarian, etc., or in a non-medical context. It is understood by anyone familiar with the art that the present invention could lower the cost of health-care, and therefore, insurance companies could cover the cost of these devices for both humans and animals.

In additional embodiments, OSWT-transmucosal extraction oral biosensors and transmucosal extraction devices are inserted temporarily or permanently in a multitude of animals. Mobile alerts are provided based on metabolic data geared toward prevention, diagnosis, and treatment of conditions, diseases, and disorders of the oral cavity, the maxillo-facial region, and its associated structures as it relates to pets, farm animals, equine, land and maritime, wild, zoo animals (A-Z), etc., are provided. Additionally, the OSWT-transmucosal extraction system of the invention includes systems and methods which interface and interact with combinations of “smart dental” and related devices. The invention incorporates one or more functions of the transmucosal extraction devices receptacle is configured to serve, e.g., is selected from the group consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible trans-mucosal extraction patch, stretch stripes, adhesive stripes, trans-mucosal extraction patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a trans-mucosal extraction gauge, trans-mucosal extraction dipstick, trans-mucosal extraction rod, trans-mucosal extraction stick, biteplate, bendable or flexible trans-mucosal extraction device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent trans-mucosal extraction and electronic for analytical and other functions and applications in order to provide accurate physiological information, etc. which can be not swallowed, with biosensors and transmucosal extraction devices on the inside of the oral cavity and, RFID tags on the outside of the oral cavity, and/or animal toys, which can be not swallowed, with biosensors and transmucosal extraction devices and RFID tag, or temporary/removable materials, used in dentistry and recreationally (e.g., tongue piercing, etc.). Biosensors and transmucosal extraction devices and RFID tags can be attached, embedded, glued, inserted, etc. to a pre-existing oral device. The transmucosal extraction and biosensors devices can be inserted by themselves into the oral tissue. This transmucosal extraction device and biosensors, when needed, can be removed by oneself at home using a piston, pulley, etc. or other devices. The oral and dental device containing the transmucosal extraction and biosensors devices can be disposable, clip-ons, stick-ons, adjustables, and/or removables. Teeth cleaning, mouth-wash, foods, or other ingested material could damage some sensors and transmucosal extraction devices, so in certain embodiments, the biosensors and transmucosal extraction devices can be protected either by removal of the biosensors and transmucosal extraction device or oral device or by covering if using airtight covers in the oral cavity itself. Using these covers and, e.g., suction devices, the sensors and transmucosal extraction devices can be cleaned or rejuvenated in the oral cavity without the need for removal. In individuals without teeth, such as babies, the device can be attached to, e.g., the gums. Patients can install, remove, and care for the transmucosal extraction devices and sensors themselves by use of moldable plastics or other moldable materials, adhesive strips, etc. The OSWT-transmucosal extraction alerting, monitoring, notification and reporting systems and other functions are configured to communicate through all application programming interfaces, e.g., cloud networks (APIs).

The following are examples of various OSWT-transmucosal extraction system communication mode classifications to which the invention is applicable: These values may be unique to each situation and to a particular patient, both human and animal, for each transmucosal extraction device application. Further, local, regional, domestic, or international monitoring and notification may be transmitted through the OSWT-transmucosal extraction system. Listed below are examples of embodiments of this invention; the ranges and preset values used here are only used as examples. It is understood by anyone familiar with the art that many more examples are possible with both humans and animals without departing from the scope of the invention. It is understood by anyone familiar with the arts that OSWT-transmucosal extraction system or transmucosal extract device along with external reader can be used interchangeably for most embodiments. However, when the reader is used the alerts will be near-time and not real-time

1. Real-Time or Near-Time Slumber to Alerts Mode.

In this mode, transmucosal extraction in real-time as diagnostic data is collected in real time, and real-time alerts are sent based on deviations from the set and/or preset values and/or ranges determined by a healthcare provider.

In one such embodiment of the present invention, a metabolic range is set or preset on a wireless device exemplified by the following scenarios: OSWT-transmucosal extraction presets a biosensors device in the mouth guard of an athlete named “Lawrence.” The OSWT-transmucosal extraction biosensor is “preset” for optimum hydration and electrolyte levels, and alerts are sent to the coach when either hydration or electrolyte levels change to set in dehydration and fatigue. Lawrence is particularly susceptible to becoming dehydrated because during recent travel he contracted stomach flu and lost body fluids. His coach can, in real time, ensure that Lawrence remains properly hydrated during his practice and games and does not incur further health damage or injuries and maintains proper athletic performance. This scenario can also be used for other human and equine sports or team sports to compare data between two or more human or equine individuals; such comparisons can assist coaches and others to ensure athlete health and performance.

In this example of an embodiment of this invention, abuse or disease in animals, children, the elderly, and the disabled is detected. Dentures for elderly, a pacifier or gum cover for babies and animals, in this embodiment of the invention, used to detect disease or abuse. Physicians and other care-takers can remotely monitor the physical and mental wellbeing of an animal or human individual. According to this aspect of the invention, a variety of biosensors may be used to detect abuse and neglect; dehydration, electrolyte imbalances, reduced nutrient levels, decreased blood glucose, altered blood medication levels, etc. are some examples of symptoms that may be monitored for signals of abuse and neglect; OSWT-transmucosal extraction in real-time or near-time with one or more of these biosensors may be used to send alerts to healthcare providers or relevant law enforcement personnel.

Chronically or otherwise sick people who need constant supervision by a healthcare provider also may benefit from this invention. For example, diabetes is a chronic illness where blood glucose levels need to be regularly monitored and controlled. When elevated blood glucose levels are observed, insulin should generally be administered to a diabetic patient to reduce and regulate the blood glucose levels; whereas, when glucose levels are reduced sugary foods should be ingested and insulin levels should be reduced. In this embodiment of our invention, insulin pumps are deployed to interface with OSWT-transmucosal extraction device so that glucose sensors and oral transmucosal extraction devices may constantly or intermittently as needed monitor blood glucose levels and dispense medication in response to blood glucose levels. Also, normal fasting glucose levels in adult humans are generally known to be between 70 and 100 mg/dL and the non-fasting value level is about 140 mg/dL. For example, when glucose levels are above pre-set value of 140 mg/dL, according to the invention an alert is sent to an insulin pump which administers a preset amount of insulin to the patient and alerts a healthcare provider. In one embodiment, the amount of insulin administered is changed remotely as needed by the healthcare provider. However, if the blood glucose levels are below 70 mg/dL, an alert may be sent to the patient, caretaker and/or healthcare provider, recommending the patient to ingest some simple sugar containing foods. For some diabetic patients, few daily routine blood glucose monitoring is required, so in this situation transmucosal extraction device combined with a “reader” with glucose biosensor can replace oral glucose sensors to provide near-time alerts.

In the above-mentioned embodiment of the invention, the glucose dispensing pump and glucose monitoring may be substituted with another transmucosal extraction device and drug dispensing pump in certain situations.

During surgery blood loss can cause severe and life-threatening dehydration, but OSWT-transmucosal extraction system can alert operating surgeon about hydration levels for the patient in real-time when OSWT-transmucosal extraction system is part of the surgical equipment.

The OSWT-transmucosal extraction system of the invention is useful in other more complex and life-threatening situations requiring more than a single biosensor and transmucosal extraction device. For example, in the treatment of congestive heart failure, cardiac arrhythmia, and post-cardiac surgery heart-attack prevention, digitalis drugs are used. The active compounds in digitalis medications are the cardiac-glycosides. Cardiac-glycosides are the most studied positive ionotropic drugs that increase the force of heart muscle contraction; strong heart contractions lead to increased cardiac output and better heart function. However, this drug has a very narrow therapeutic window, so slight increases in blood glycoside levels may cause the drug toxicity to manifest itself in severe and irreversible side effects, which may even result in death. Additionally, glycosides are very sensitive to blood potassium levels, and low potassium levels enhance the drug's side effects. In this embodiment of the OSWT-transmucosal extraction system of the invention, real-time or near-time OSWT-transmucosal extraction with biosensors for glycoside and potassium is placed in the patient's mouth and alerts are sent to a healthcare provider and/or patient if either one or both blood glycoside and/or potassium levels reach near toxicity levels. The alerts may lead to a reduction in digitalis and or potassium doses. This example of an embodiment of the OSWT-transmucosal extraction system of the invention may shorten the hospitalization of post-surgery patients who are on digitalis, and thus, significantly reduce the cost of their care. In this embodiment of the inventions, any two or more drug interactions may be determined and the OSWT-transmucosal extraction system may communicate with different nano-particles in the drugs.

Another embodiment of the OSWT-transmucosal extraction system of the invention uses several biosensors and transmucosal extraction devices and other devices located on or near the animal or human. Cisplatin is known to be used in the treatment of lung, testicular, bladder, and ovarian cancer. Unfortunately, this drug causes severe side effects, including hypertension, ischemia, and atrial fibrillation. This situation is a complex one because the drug may cause several life-threatening side effects that need to be monitored simultaneously with the patient's blood cisplatin concentrations. In this embodiment, an OSWT-transmucosal extraction system is installed with biosensors and transmucosal extraction devices for cisplatin concentration detection. Cisplatin concentration detecting biosensors and transmucosal extraction devices can be coupled with one or more of the following biosensors: blood pressure for hypertension, oximeter for ischemia, and heart rate for fibrillation. A preferred diagnostic tool for atrial fibrillation is a cardiac monitor hooked to the chest of a patient who constantly or frequently monitors heart function. Therefore, in this embodiment, these patients are optionally hooked up to a cardiac monitor. The OSWT-transmucosal extraction system described herein sends alerts to a healthcare provider and/or to a drug dispensing pump whenever the patient suffers from one or more side effects for which intervention is indicated. The alerts may optionally alter drug dosage to alleviate the side effects. Additionally, the OSWT-transmucosal extraction system of the invention optionally contains other drug dispensing pumps or devices that administer additional medications to alleviate side effects. For example, pumps to dispense antihypertensive or anti-arrhythmia medications are a potential part of the OSWT-transmucosal extraction system described herein. A defibrillator device is also optionally a part of the OSWT-transmucosal extraction system to treat and eliminate cardiac fibrillation. Because electrolyte imbalance may result from treatment with cisplatin, alerts about such electrolyte imbalances can be bi-directionally transmitted to a physician who can further check the patient for possible infections. This example exemplifies how the OSWT-transmucosal extraction system may use multiple routes to diagnose one or more problems and treat several symptoms simultaneously and in real- or near-time. In this embodiment, it is possible that the OSWT-transmucosal extraction system of the invention communicates with the drug dispensing pump, several medical devices, and nano-particles in the medication to get a very accurate overall health picture of a patient, and this overall picture is important to save a life.

Furthermore, if tracers with nano-particles or a customized chemo-medication are routinely used to is used during surgery to either trace or destroy cancerous cells. Because these medication formulations need to be administered carefully such that their concentrations in blood are maintained within a narrow therapeutic range, communication between the drug dispensing pump, blood medication measuring sensors, and measurements potential side-effects are important. In this embodiment, the OSWT-transmucosal extraction system of the invention may communicate with both a transmucosal extraction device pump and nano-particles in the medication to determine an exact and accurate blood drug concentration.

Severely sick people often suffer from more than one illness. Therefore, they are frequently administered several medications that interact with each other changing their effects and causing several additional side effects. For example, a person suffering from cardiovascular disease with high cholesterol levels while also being treated for a cancer, may be receiving statin drugs to reduce cholesterol levels, which may render cancer chemotherapy being received more potent by increasing the blood concentration of the chemotherapy drug, thereby enhancing the potency and toxicity of the chemotherapy agent. In this embodiment, the OSWT-transmucosal extraction system of the invention utilizes biosensors for the following: statin drug or nano-particles in statin concentration detection, blood lipid profile or cholesterol concentration detection, and the chemotherapy drug such as cisplatin or nano-particles in cisplatin concentration detection, could help determine the exact amount of chemotherapy drugs and statin drugs being administered to treat both diseases simultaneously.

Animal research forms an integral part of basic and pharmaceutical research. These researchers may use the OSWT-transmucosal extraction system herein to collect more data from the research animals, and therefore, reduce the number of animals needed for use in their research. In one embodiment of the OSWT-transmucosal extraction system as described, a scientist discovers a new pathway to treat diabetes by inhibiting a function of a protein X, and he wants to test a number of X inhibitory compounds. In cell models, compounds #1205, 1252, 0501, and 0512 were shown to be good inhibitors in descending order, decreasing strength with 1205 being the strongest and 0512 being the weakest inhibitor. Thus, he now wants to test these compounds for their bioavailability and toxicity in animals and decide which compound is the best candidate for the treatment of diabetes in humans. Unaware that compound #1205 had very limited bioavailability and caused a dramatic decrease in blood calcium levels through an unknown pathway. He checks blood levels of the drug every one hour and blood chemistry every 24 hours after drug administration in mice. However, the decreased blood calcium had already returned to normal before the first 24 hours calcium measurements because of calcium in the diet, so he has missed a very severe and life-threatening side effect. In fact, unknown to him, compound #0501 is the best candidate because it had the least side effect profile. After administering the drug for a week, he sacrificed the animals to investigate effects of the drug on various organs, but the heart tissue in these animals had already recovered. Therefore, these experiments were wasteful, and the scientist did not realize that the animals had suffered. In this example, the drug could not get FDA approval even though the new pathway was very encouraging. However, if real-time OSWT-transmucosal extraction according to the invention used had been available to the scientist, he would not have missed the window of severe side effects and would have chosen compound #0512 for further drug development. In this scenario, this compound could have led to the development of a new diabetes drug which potentially could have been very helpful many humans and other animals suffering from diabetes but was tragically missed. This embodiment of the invention is designed to help avoid such current gaps in research and development, as well as mitigate unnecessary overuse of test animals.

In yet another embodiment of this invention, nano-particles are used to diagnose cardiac function and blood flow related diseases such as, but not limited to, obstructions in blood vessels caused by plaques which are precursors of atherosclerosis and stroke. Nano-particles such as dyes, fluorophores, etc. can be injected, in the arm, for example, and the movement of the nano-particles through the vessels and heart can be followed and communicated through the OSWT-transmucosal extraction system. This can be done in real life when the person is doing daily activities, and not be limited to a hospital setting with imaging equipment. Following daily life for longer periods is more useful in several medical conditions. Various blood vessel and cardiac functions can be calculated by complex mathematical equations known to, or which can be developed by, one skilled in this art. A plurality of sensors, transmucosal extraction devices, and biosensors RFID or other micro- and nano-communication and energy harvesting, micro- and nano-energy storage components may be installed in any dental device and/or placed in an oral cavity, depending upon the medical or diagnostic intent. In addition, in other embodiments, the OSWT-transmucosal extraction systems of the invention may be used in conjunction with other smart wearables, attachables and insertables anywhere on the body of the same or another individual to form a network of smart wearables or other medical devices used by a collective group such as a basketball or other sports team. A single or a set of wearable or other medical devices can be placed on the team member/s that send alerts and communications, through the OSWT-transmucosal extraction system in real-time or near-time, to the coach and/or the team's healthcare provider, to ascertain the medical and/or diagnostic condition of each team member for the best sports outcome. The data collected may be used to determine the health status of each individual and/or to compare with another team member or collectively as a team. For example, a player might be very important to the successful outcome of the game but he is injured; based on his bodily functions as determined by the OSWT-transmucosal extraction system, his performance can be predicted and the information used to help the coach to make strategic decisions. In another example, the coach compares statistics between two or more players on the team to make a decision about their performance.

The OSWT-transmucosal extraction system can make available the ability to integrate data concerning numerous team and individual sports models as exemplified by the following definitions:

2. Manual Control Diagnosis Mode.

In addition to the above, the OSWT-transmucosal extraction system user, caretaker, healthcare provider, and others are able to manually activate one or more biosensors and transmucosal extraction device data-points in order to visualize and analyze information inputs from the host animal or human.

Nina wants to lose weight and needs to reduce her blood glucose, triglyceride, and cholesterol levels. However, due to her traveling job, she finds it difficult to regulate her food and caloric intake. When she is traveling every morning and after every meal, she activates her OSWT-transmucosal extraction system of the invention with biosensors for blood sugars, total blood lipids, triglyceride levels, cholesterol levels, and blood proteins and/or amino acid levels. The values obtained from these biosensors along with appropriate mathematical calculations known to those skilled in the art could assist the OSWT-transmucosal extraction system to compute the number of calories she consumes at every meal. Her fasting blood component levels helps her in closely monitoring on her blood glucose, total blood lipids, triglyceride levels, and cholesterol levels. The described OSWT-transmucosal extraction system sends Nina daily reports with a breakdown of her calorie and dietary consumption. When she has consumed the required intake of calories and/or other nutrients, she is alerted to change her remaining meal composition to reduce additional lipid or sugar or high caloric food intake, or to exercise to burn off extra calories. In this embodiment of the invention, the OSWT-transmucosal extraction system may communicate with other smart devices and apps such as Fitbit, etc. Weight gain and obesity result from imbalances in food and caloric intake and caloric output, e.g., by exercise, yet most programs, such as Fitbit, only address caloric output. Biosensors and transmucosal extraction devices used according to an embodiment of the invention can help establish healthy eating habits by evaluating food intake habits, and analysis of ingested food for weight loss and maintaining of healthy levels of blood glucose, total blood lipids, triglyceride levels, cholesterol levels, etc.

3. Programmed Automated Diagnosis Mode.

In addition to the aforementioned modes, the user of the described OSWT-transmucosal extraction system can automatically activate one or more biosensors and transmucosal extraction device data-points in order to visualize and analyze information inputs from a host animal or human. In this embodiment, the OSWT-transmucosal extraction system is set to any time schedule (second, minute, hour, day, week, month, etc.) in order to activate or monitor the medical health of any species or patient.

Additionally, general symptoms, such as body hydration level, electrolyte balance, blood glucose levels, protein-C, hemoglobin A1C, temperature, and/or heart rate, optionally may be monitored to predict early onset of various chronic diseases, infections, or emerging diseases. Specific symptoms, such as elevated glucose and hemoglobin A1C that predict diabetes may be monitored regularly. In one embodiment of the invention, blood glucose levels are routinely monitored. Fasting blood glucose levels monitored during early morning before food ingestion are the best predictor of diabetes, and these daily monitoring may be programmed. Normal glucose levels between 70 and 100 mg/dL and levels above 100 mg/dL send an alert to the healthcare provider. Hemoglobin A1C levels in the body reflect an increase in blood sugar for two to three months, and thus, these should be measured approximately once every one or two months. Hemoglobin A1C levels above 4% to 5.9% may be set to cause the described OSWT-transmucosal extraction system to send an alert to the healthcare provider.

In a further embodiment, the specific measurements of a symptom that helps predict any of a variety of diseases are set on alert. For example, elevated levels of C-reactive protein are an inflammation predictor. C-reactive protein could be a symptom of a variety of diseases such as metabolic diseases, infections, etc. In one embodiment of the invention, blood C-reactive protein levels are routinely monitored, and when the levels are above a preset value, alerts are sent to the healthcare provider who could further investigate the cause of this increase.

In yet another embodiment of this invention, the described OSWT-transmucosal extraction system is used to help the sick who are prone to fluctuation in other symptoms such as electrolyte imbalances. For example, alterations in blood concentrations of sodium or potassium are predicting a variety of diseases such as kidney diseases, malnutrition, dehydration, and loss of fluids by either vomiting and/or diarrhea. Monitoring medications for appropriate alteration in their concentrations are also possible for healthcare providers who want to regularly monitor their levels for some patients using the described OSWT-transmucosal extraction system. When such imbalances are alerted, the healthcare provider is able to intervene to correct the imbalance.

4. Geographic Analysis Mode.

In addition to the above, in a further embodiment, a geographical OSWT-transmucosal extraction system dissemination and locator mode is also utilized in order to, for example, monitor and analyze a herd of cattle, a herd of sheep, or other animals on a protected geographical range.

Certain diseases can be linked to a local diet, environment, cultural factors, disasters, etc., and the described OSWT-transmucosal extraction system may be applied in these situations. For example, the nuclear spill, such as the one due to an earthquake, in the Kashiwazaki-Kariwa Nuclear Power Plant in Japan, caused humans and animals to be exposed to high levels of radiation. Exposure to high levels of radiation causes a variety of cancers. Early detection of cancer enhances the prospects for a good outcome of most cancer treatment. In one embodiment of this invention, a panel of various cancer biosensors is placed every six months, in the oral cavity of the exposed people, and the OSWT-transmucosal extraction system alerts healthcare providers about the development of new signs of cancer in these individuals.

In order to increase the number of critically endangered animals, females that become of child-bearing age must be kept in good health. In one embodiment of this invention, these females of child-bearing age are connected to the OSWT-transmucosal extraction system with biosensors for testing positive pregnancy. For example, Rhinos in Sumatra are endangered, so all female Rhinos of child-bearing age may be connected to an OSWT-transmucosal extraction system with biosensors for hormones and other biologics that change during for pregnancy can be used for testing positive pregnancy. When alerts of pregnancy are received, the authorities take measure to enhance or ensure the good health of the mother and that healthy baby Rhinos are born by providing the mother with appropriate nutrients and medication in the wild. This could increase the Sumatran Rhino population. A similar system can be used in horses to determine pregnancy so that proper can be provided. Pregnancy in horses can be determined within 30-40 of pregnancy via blood draw, whereas, other methods can take up to 90-120 days. OSWT-transmucosal extraction determines pregnancy much earlier than other available non-invasive methods currently available.

Training and performance of human and equine athletes are impacted by geographical locations. OSWT-transmucosal extraction devices as described herein can be designed to outfit humans as well as animals. During athletic activities heat loss and electrolyte loss are closely linked to geographical area and local weather; therefore, based on a location associated performance and medical diagnostics such as, but not limited to, hydration, and electrolyte levels, a protocol is established which greatly assists the athlete's or equine's training and performance. For example, a human or horse athlete can loss more heat, hydration, and electrolytes more easily in the arid climate of Saudi Arabia as compared to winter months in French Alpines. Therefore, diagnostics and performance data is the difference in a same individual athlete but in a different region. A historical comparison with past performances made during different season and locations will enhance training and performance. Thus, the OSWT-transmucosal extraction system mode of the invention is important to help determine proper precautions which enhance the optimum performance under various geographical, medical, and performance conditions.

5. Species Classification Analysis Mode.

In a further example, a specific endangered species is monitored through the described OSWT-transmucosal extraction system network. Animals on the verge of extinction would benefit from this invention. In addition to monitoring the animal's survival, the transmucosal extraction devices and biosensors also are capable of monitoring them for their disease, nutrition, and overall health status. Therefore, appropriate intervention becomes available to increase their life-span and reproduction. Overall, these transmucosal extraction devices and biosensors reduce the risk of extinction for several animals. Many different embodiments of this invention are possible with both humans and animals.

This mode of the invention is specifically important because not all animals have the potential of developing the same diseases. Dogs, for example, could become ill from ingesting onions and chocolate, which in turn are very beneficial for humans. Cats, unlike dogs, but like humans, develop metabolic diseases easily and at a younger age, such as diabetes, cardiovascular problems, and other obesity-related diseases. Pathogens for tooth and gum infections are different in pet animals than in humans. Several pathogens are species-specific, meaning that they cause disease in one species but not in others. For example, Coronavirus (SARS-CoV) cause severe acute respiratory syndrome (SAR) in humans, but not in other animals such as domesticated cats.

6. Specific Disease or Situational Alerting Mode.

In addition to the aforementioned modes, the OSWT-transmucosal extraction system of the invention is a source of disease-specific or situation specific modes of operation.

Mary is busy traveling for her job. Therefore, she misses her doctor's appointment. But her doctor sends her a packet with several biosensors and instructions to use them (according to the invention). The panel of biosensors sent is based on her medical history, her family medical history, and any diseases specific to her. Examples of biosensors coupled to transmucosal extraction devices included in the packet for use in the invention include blood chemistry sensors such as sodium, potassium, calcium sensors, blood glucose, lipid profile sensors, hemoglobin, hemoglobin A1C, blood creatinine, cortisol, and C-reactive protein, temperature, heart rate, blood pressure, cancers that run in her family, common infections and allergens found in her environment, and any drugs that she might be prescribed at the time. She uses each biosensor as instructed; such as, blood glucose should be used during early morning fasting time, but cortisol should be used in evening hours; whereas, the temperature one should preferably be used at all hours for over a few days. All values obtained are sent to her physician's office, and her physician may discuss her health status with her during her next visit.

7. Activation and Wake-Up by another Biosensor, Transmucosal Extraction Device, Device, and/or Remote Mode.

The OSWT-transmucosal extraction system described herein includes biosensors and transmucosal extraction devices located elsewhere within and/or on an animal or human. The OSWT-transmucosal extraction system also interfaces with various other devices located on or near an animal or human, and may securely communicate with a plurality of remote devices that monitor the health and/or well-being of one or more animals or humans. Other diagnostic devices, drug-dispensing devices, other devices, and/or other biosensors and transmucosal extraction devices are optionally used to activate or wake up the OSWT-transmucosal extraction system of the invention. The OSWT-transmucosal extraction system, e.g., may also is optionally remotely woken up by a caretaker, healthcare-provider, and/or others. In yet another embodiment of the invention, the described OSWT-transmucosal extraction system triggers activation and wake-up. These triggers for example, in certain protocols, includes the oral cavity's tongue/teeth/finger(s), etc. and maybe self-induced in order to control and activate one or more biosensors and transmucosal extraction devices within the oral cavity in conjunction with remote activation through one or more smart devices.

An example of a diagnostic device that may be used in this system, a cardiac monitor is employed to detect abnormalities. When it does so, it sends a signal to activate the described OSWT-transmucosal extraction system to begin monitoring and collecting data about the patient's electrolyte imbalances, blood pressure, and/or prescribed drug blood concentrations. The cardiac monitor also sends an alert to the healthcare provider who may remotely activate additional biosensors within the OSWT-transmucosal extraction system. Another example of drug-dispensing device activation involves when a cisplatin-dispensing pump activates the OSWT-transmucosal extraction system to monitor blood pressure, EKG, electrolytes concentrations, and/or blood cisplatin concentrations. If one or more of these values are sufficiently different from normal preset values, the OSWT-transmucosal extraction system may send an alert to the drug-dispensing pump to alter the amount of drug being administered. An alert is also optionally sent to the healthcare provider, so they take any appropriate action. In this embodiment, the OSWT-transmucosal extraction system of the invention may communicate with medical devices, a drug dispenser pump, and nano-particles as to the medication for accurate diagnosis and treatment.

8. Communication through WiFi and other Wireless Modes.

In this mode, WiFi, GPS, GPRS, or other secure communications occur between the described OSWT-transmucosal extraction system and sensors and transmucosal extraction devices located elsewhere within and/or on an animal or human. The OSWT-transmucosal extraction system also securely communicates through WiFi, GPS, GPRS, or other methods with other devices located on and/or near an animal or human. These communication modes are subject to a plurality of remote devices that monitor the health and/or well-being of one or more animals or humans.

James is an elderly person who lives alone. His nurse visits him weekly to monitor his vitals and other medical symptoms to ensure his well-being. In this example, James suffers from several diseases. James provides the OSWT-transmucosal extraction system kit contains several disease-specific biosensors included in his dentures to diagnose problems with his overall health and disease status. He is provided either with a smart necklace or a smartwatch that he is supposed to wear at all times. These smart accessories contain a built-in WiFi and/or GPS, GPRS, microphone, and speakers. The OSWT-transmucosal extraction as described could be remotely turned on by the nurse or physician to measure specific biosensors when the blood, stool, or urine collected weekly by the nurse suggests abnormal levels of a biologic. This further saliva testing could ensure that James is doing well and his medication dosing could be changed before next visit by the nurse. In this example, certain circumstances could save James live and at the same time reduce healthcare cost.

9. Concentration Alert.

In this mode, a component from blood supply in the oral cavity and/or lymph nodal fluids is optionally concentrated; when sufficient preset amounts of the said component are concentrated, an alert is sent and the biosensors along with the said component may be removed for further analysis. Encapsulated or un-encapsulated biosensors may act as an isolation and/or concentrating device with alert and communications sent at appropriate times. These communication modes are subject to a plurality of remote devices that monitor the health and/or well-being of one or more animals or humans.

In one example of this embodiment of the invention, biosensors are unable to determine the exact sub-type of cancer cells present in oral blood supply. Transmucosal extraction device with biosensors for all types of tumor, which represent 0.01% of all cells in the blood, is installed on a retainer in the oral cavity. The biosensors bind to several types of tumor cells based on cell-surface biomarkers and only 10% of these tumor cells are malignant and cancerous. Therefore, blood fluid might contain 0.001% or less of malignant cells needed to diagnose cancer. To collect ten microliters of the malignant cells for analysis in the laboratory, 100 milliliters or 0.1 liters of blood fluid might be needed. However, these cells do not survive for too long outside of the body; so when outside of a human body, these cells need to be stored properly at subzero temperatures. Due to these conditions, it is difficult to diagnose these cancers accurately until malignant cell concentrations in the blood increases; however, when more such cells are present in the blood the disease could have progressed and could lead to poor prognosis for the patient since by then the disease might have advanced and be difficult to treat. However, according to this aspect of the invention, the biosensors are allowed to bind to the tumor cells for several hours and even longer, whereby a sufficient preset amount becomes concentrated to properly, reliably, and timely diagnose cancer. When a sufficient preset amount binds or absorbs to the biosensors, an alert is sent to remove and send the biosensors data acquired for further analysis. This situation can be used according to the invention wherein it is substituted by other infectious diseases or other diagnostic molecules that are initially present in sub-diagnosable amounts.

In another example of this embodiment, still undiscovered, specific RNA is a predictor of a certain metabolic disease. However, the amount of this RNA in blood is so minuscule that it makes the RNA identification difficult or even impossible, so the disease prognosis is poor. In this aspect, the biosensors of the OSWT-transmucosal extraction system of the invention concentrates this RNA over the course of several hours or longer until the RNA in question is sufficiently concentrated and an alert is sent to remove the OSWT-transmucosal extraction and biosensor device, or to send its accumulated data, for further analysis. This ability to concentrate might not be practical by commonly practiced RNA concentration methodology from blood due to the minuscule amount of RNA present in the blood. The RNA containing biosensors according to this aspect of the invention can then be sent, or its accumulated data sent, to a laboratory for analysis, for example, by amplification, sequencing, etc. This entire process makes diagnosis more reliable and accurate.

10. Other Combinations Using the OSWT-Transmucosal Extraction System.

In addition to the above, any and all combinations could be exemplified for a multitude of OSWT-transmucosal extraction purposes and uses. An auto-mode, manual mode, etc. pertain to multiple OSWT-transmucosal extraction applications.

This example may be utilized for both human and animal patients. This embodiment reflects a combination of all communication modes for described OSWT-transmucosal extraction system. The utility of this example of an embodiment in WiFi modes is previously explained in detail. The OSWT-transmucosal extraction system is programmed to become automatically activated when a disease or elevated stress levels are detected as previously exemplified. The OSWT-transmucosal extraction system of the invention may be used for any breed, pet, or for farm animals, such as horses, cats, dogs, and cattle. Further laboratory animals could be subjects of the species-specific embodiment of the OSWT-transmucosal extraction system. The OSWT-transmucosal extraction system is optionally used for any metabolic disease, other diseases, and/or situational use; so this embodiment can be disease and/or situational specific. The owner, caretaker, and/or another person, remotely or otherwise, activate this embodiment in manual mode. The OSWT-transmucosal extraction system of the invention enables oral sensors to interface with other devices located on or near an animal or human, and securely communicate with a plurality of remote devices monitoring the health and/or well-being of one or more animals or humans. Thus, this embodiment can be used as a OSWT-transmucosal extraction device or another biosensors and with remote activation or wake-up mode settings. Caretakers, healthcare providers, etc. can visualize data such as, but not limited to, correlations between different episodes and historical data any animal, or human within the same species, breed, or disease category, or within different species, disease categories, or situations.

In still another embodiment of this mode of the invention, biosensors can be used to determine fertility, ovulation, and pregnancy. The OSWT-transmucosal extraction device can be set to a combination of automated, real-time or near-time and manual modes. It is understood by anyone familiar with the art that in females, basal body temperature and several hormones associated with menstrual cycles and pregnancy change during the menstrual cycle. For example, during the ovulation phase of the menstrual cycle, follicle stimulation hormone (FSH) and Leutenizing Hormone (LH) from the pituitary glands are elevated. During pregnancy, the pregnancy announcing hormone, human chorionic gonadotropin (hCG), appears elevated in the blood and during several labor hormones, including oxytocin, adrenaline, and endorphins become elevated. Biosensors for various aforementioned hormones may help in the diagnosis of the onset of ovulation, pregnancy, labor and some diseases related to fertility by use of real or near time, manual, and programmed automated alerts. Accordingly, incorporating such bio-sensing indicators into the OSWT-transmucosal extraction device of the invention provides for the benefits delineated above.

In another embodiment of the invention, Sara visits her physician for a routine check-up, but she does not want blood drawn, so she is given a dental device with OSWT-transmucosal extraction device. Before she goes for her check-up, her physician sends her a package with OSWT-transmucosal extraction dental device and instructions for use. It contains OSWT-transmucosal extraction device for cortisol and glucose with re-programmed and real-time modes for OSWT-transmucosal extraction device. She wears the device morning and evening, and the results could be sent to her physician. At the clinic, she wears to the device to measure her electrolytes in real-time while the device is in her oral cavity. However, uric acid or urea and other disease-related DNA could be measured in near-time by a reader. Since Sara has a history of periodontal disease and cytomegalovirus infections, so these would be sent to a laboratory for the micro-organism growth and testing.

According to embodiments of the invention, different tier, degree, and intensity levels of alerts are possible with each one of the alert modes. Basal measurements for any diagnostic parameter can be set at the lowest level of alert for each mode. Some alerts can be established as repetitive alerts, and bi-directionally transmit out alerts at pre-set interval until alert-receipt is acknowledged by the receiver.

OSWT-transmucosal extraction -related biomarkers and applications: these transmucosal extraction systems with biosensors optionally are used for a variety of athletic performance- and disease-related biomarkers including, but not limited to, oral and systemic infectious diseases, cancers, drug-monitoring, dehydration levels, electrolyte levels, metabolic diseases, malnutrition, obesity, cardiovascular diseases, atherosclerotic inflammation, stroke, and still-to-be-discovered disease- and athletic performance-signature and disease- and athletic performance-linked biomarkers. While all of these and other applications, with a variety of transmucosal extraction devices with sensors and biosensors, would yield significant information, the information without the communication and alert systems would not be nearly as useful to human and animal patients, caretakers, healthcare providers, and or others. This communication and alerting system, along with transmucosal extraction and biosensors devices, are incorporated into the invention OSWT-transmucosal extraction system and transmucosal extraction along with the reader. In addition to the transmucosal extraction devices with biosensors, the OSWT-transmucosal extraction system of the invention includes other biosensors and transmucosal extraction devices that interface with the OSWT-transmucosal extraction system, and are located elsewhere, within, and/or on an animal or human. The OSWT-transmucosal extraction system is capable of interfacing with a variety of other devices and nano-particles that are located in or on or near the animal or human, and can securely communicate with a plurality of remote devices that monitor the health and/or well-being of one or more animals or humans. The OSWT-transmucosal extraction system of the invention also includes secure data system software with visualization modes and presentation system software. The latter feature is available to utilize a variety of platforms including, but not limited to, charts, graphs, histograms, and/or bar graphs. Caretakers, healthcare providers, etc. can visualize data including, but not limited to, correlations and comparisons between different episodes and historical data for patients within the same species, breeds, and/or disease categories; or within different species, disease categories, and/or situations. For example, data from laboratory animals can be correlated and extrapolated to other animals or humans suffering from the same or similar diseases. Such kinds of scenarios are possible because the secure data system contains data captured and/or collected from various alerts. The secure data system also inputs data into the system in order to set alerts, which can be analyzed and visualized by the OSWT-transmucosal extraction system software. Some of this data will be viewable and available to everyone, so will be public, yet some data will be viewable and available only to relevant individuals and healthcare providers, and hence will be private. The secure data system also inputs data into the system in order to set alerts, which can be analyzed and visualized by the OSWT-transmucosal extraction software according to this aspect of the invention.

Divergent applications are possible with these transmucosal extraction devices with biosensors. The device may be present in an oral cavity for varying amount of times. The device is optionally part of a plethora of oral and dental devices and/or it can be implanted flexibly, adhered, fixed, and/or cemented to a variety of locations within the oral cavity. Specifically, the device is securable inside of or affixed to the cheeks, tongue, sublingual cavity, and/or other places within the oral cavity such that they are not swallowed. The device locations within the oral cavity are optionally chosen by the patient, caretaker, and/or healthcare provider based upon convenience, comfort, need, duration of placement, durability, and manufacturing ease, and regulatory guidelines, and other factors. The biosensors and transmucosal extraction device might be placed by a healthcare provider including, but not limited to, physician, surgeon, dentist, veterinarian, healthcare provider assistant, caretaker, and/or the patient him- or herself; the placement might be done, e.g., no sedation, under mild sedation, full sedation, local anesthesia, general anesthesia, or other.

The oral cavity is supplied by ample blood which reflects systemic blood, so changes in the molecular composition of the oral blood are consequently reflective of systemic blood. Therefore, disease-specific blood-based biomarkers in oral cavity blood depict the biomarkers of the entire body and systemic system. These biomarkers are also used to diagnose and monitor systemic diseases. Often these biomarkers precede disease developments in both humans and animals; thus, these biosensors and transmucosal extraction devices help determine the overall health statuses of both animals and humans. The oral cavity is unique in preventing several disorders or treating them in the early disease stages. Thus, this invention can help save billions of dollars in healthcare costs and increase both quality and quantity of life.

Biomarkers are found in blood, interstitial fluids, and lymph node fluids. The National Institute of Health (NIH) defines a biomarker as any objective, measurable, and evaluable indicator of normal biologic processes such as diseases, disorders, athletic performance, etc., pathogenic processes, or pharmacologic responses to therapeutic interventions. A biomarker that is reproducible or consistent, reversible, and reliable and may be associated with a particular disease is a signature of that disease. A variety of molecules can be biomarkers including, but not limited to, proteins, immune response molecules like immunoglobulins and antibodies, nucleic acids like DNA and RNA, lipids, metabolites, histones, modified proteins and nucleic acids, salts, cations, anions, and microbes. Alterations in signature biomarker concentration, structure, function, and/or action could be associated with the onset, progression, or even regression of a disease, optimum health, and athletic performance associated with the signature biomarker. Therefore, the biomarker is a valuable tool in the detection, diagnosis, risk assessment, prognosis, and monitoring of a disease.

Lymph nodes offer new, novel, and unique opportunities to detect several diseases where the immune response is the initial primary response, or where biomarkers are concentrated in the lymph fluid. Examples of such diseases are infections, certain cancers, ingested pathogens, allergens, and toxins. Oral secretions drain into three lymph nodes—the submandibular, submental, and tonsillar fluids, from the cheeks, the lower lip, gums, and the anterior tongue, drain into the submandibular node from the lower lip and mouth floor. Fluids from the tongue apex drain into the submental node. Fluids from the jugulodigastric drain into tonsillar lymph nodes. Biosensors and transmucosal extraction devices implanted in these lymph nodes according to the invention helps in early diagnosis, detection, risk assessment, prognosis, and monitoring of a variety of diseases with immune responses, infections, and locations where biomarkers either concentrate or appear during early disease progression.

The described OSWT-transmucosal extraction system is utilized, as described above, by animal and humans such as infants, children, disabled, or the elderly who are unable to communicate their health status to their caregivers. These individuals especially benefit from employment of these biosensors using the OSWT-transmucosal extraction system of the invention. Besides predicting disease amongst humans and animals, they optionally are employed in determining athletic performance and abuse. Athletic performance can change dues to several bio-molecules such as electrolyte, hydration levels, blood glucose, etc.; whereas, abused animals are often malnourished, injured, and dehydrated. Hence, malnutrition, injury, electrolytes, dehydration, and others may be assessed, detected, diagnosed, and monitored for signs of both athletic performance and abused humans and animals.

Data collected from animals using the described OSWT-transmucosal extraction system not only helps determine their health status but also may optionally be extrapolated to humans. These animals could, in other words, constitute and serve as animal models for humans. Every disease has a panel of diagnostic testing, recommended by healthcare organizations such as Center for Disease Control (CDC) or Center for Medicaid and Medicare (CMM). Problems associated with diagnostic testing are major hurdles to early detection and effective treatment. Most diagnostic tests are expensive, time-consuming, invasive, and inaccurate. Therefore, researchers are constantly searching for ways to improve current diagnostic tests or invent new methods of diagnostic testing. Timely diagnosis is an important key to the proper treatment of any disease. Transmucosal extraction devices along with biosensors used according to the described invention offer new, innovative, and unique opportunities to diagnose, detect, and monitor diseases.

The OSWT-transmucosal extraction system of the invention offers a timely, inexpensive, non-invasive or minimally invasive, accurate, and consistent diagnostic tool to monitor a variety of therapeutic, athletic performance, diseases and pathophysiological conditions in animals and humans. It eliminates costly, invasive, painful, and time-consuming diagnostic testing methods. The invention's use of technologies incorporating wearable devices, devices capable of storing and analyzing large amounts of electronic data, and wireless batteries enhance the benefits provided by the OSWT-transmucosal extraction system of the invention. Frequent and continuous monitoring of a large amount of data generated is readily maintained, analyzed, and handled by the inventive system and method. Moreover, the wireless charging utilized is beneficial when frequent or continuous monitoring is required.

It is to be understood that the above description is intended to be illustrative, and not restrictive. For example, the above-described embodiments (and/or aspects thereof) may be used in combination with each other. In addition, many modifications may be made to adapt a particular situation, method, system device or material to the teachings of the various embodiments of the invention without departing from their scope. While the particulars and details described herein are intended to define the parameters of the various embodiments of the invention, the embodiments are by no means limiting and are exemplary embodiments. Many other embodiments will be apparent to those of skill in the art upon reviewing the above description. The scope of the various embodiments of the invention should, therefore, be determined with reference to the appended claims, along with the full scope of equivalents to which such claims are entitled.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims and may include other examples that occur to those skilled in the art. Such other examples are intended to be within the scope of the claims if the examples have structural elements or steps that do not differ from the literal language of the claims, or if the examples include equivalent structural elements or steps with insubstantial differences from the literal language of the claims.

We claim:

1. A device comprising a trans-mucosal extraction receptacle for blood chemistry analysis which is securely placed within a human or an animal's oral cavity without being swallowed.

2. The device of claim 1 wherein the at least one trans-mucosal extraction device comprises of electrical, electroporation, ionophoresis, reverse ionophororesis, thermal ablation, laser, radio frequency heating, chemical heating, mechanical heating, vibrational, ultrasound, sonophoresis, phonophoresis, mechanical, micro-needles, tape-stripping; chemical-peels, enzyme peels, chemical enhancers, surfactants, liposomes, microemulsion, niosomes, proniosome, carrier-associate vesicles or drugs, enzyme inhibitors, nanoparticles, polymer-chain associated drugs, microparticulate, mucoadhesives, diffusion, osmosis, electro-chemical gradient assisted devices, electro-chemical gradient based, salt, natural-made polymers and compounds, man-made polymers and compound, diffusion-assisted, enzymes, polymer-chain, microparticulate, velocity-assisted devices, powder injections, jet injections and any combination thereof.

3. The device of claim 2 wherein the at least one part of transmucosal extraction device is in contact with oral tissues.

4. The device of claim 2 wherein the device further comprising of at least one biosensor interacts with at least one disease-related biomarkers from blood, interstitial fluid, lymph node, bone, or tooth constituent selected from the group consisting of oral and systemic infectious diseases, cancers, drug abuse, metabolic diseases, malnutrition, obesity, cardiovascular diseases, atherosclerotic inflammation, stroke measure a predetermined biologic, measure a predetermined biologic including or consisting of DNA, RNA, telomeres, methylated or otherwise modified DNA or RNA, proteins, immunoglobins, antibodies, histones, peptides, modified proteins, neuro-peptides, pigments, and enzymes, measure dissolved gases, including oxygen, carbon dioxide, carbon monoxide, ammonia, sulphur, an alcohol-containing gas, measure a lipids profile, measure a chemical molecule, measure a salt, an alcohol, a metabolite, an anion, a cation, water, a sugar, a protein, a lectin, measure a drug, a medication, measure cells, cancerous cells, biomarkers for drug abuse, biomarkers for a metabolic disease, biomarkers for malnutrition, biomarkers for obesity, biomarkers for a cardiovascular disease, biomarkers for atherosclerotic, biomarkers for infection, biomarkers for auto-immune, other immune diseases, biomarkers for stroke, biomarkers for AIDs, biomarkers for multiple sclerosis, biomarkers for periodontal diseases, biomarkers for brain-function disorders, dementia, memory loss, depression, mental disease, Alzheimer's disease, mentally-challenged disorders, nervous system disorders, tracking or wandering, and other psychology and neurological disorders, biomarkers for bleeding, head and neck injuries, biomarkers for Sjogen's syndrome, biomarkers for oxidative stress, biomarkers for allergies, biomarkers for cancer, biomarkers for skeletal and muscle diseases, biomarkers for genetic diseases, biomarkers for renal diseases, biomarkers for osteoporosis, biomarkers for fatigue, biomarkers for stress, biomarkers for sleep deprivation or sleep apnea, biomarkers for fertility, pregnancy, ovulation, and reproductive system disorders, biomarkers for cystic fibrosis, biomarkers for respiratory or pulmonary diseases, biomarkers for diabetes and ketoacidosis, biomarkers for inflammation, biomarkers for age-related diseases, biomarkers for dehydration, biomarkers for halitosis, biomarkers for alcohol consumption, alcoholism or drug consumption or drug addiction, biomarkers for hypoxia, smoking-related diseases, toxins, or pollutants, biomarkers for poor-gait, biomarkers for Crohn's disease, biomarkers for dental caries, biomarkers for blood and circulatory disorders, biomarkers for ear, nose, and throat diseases, biomarkers for taste, Ageusia, Hypogeusia, or Dysgeusia, biomarkers for bad-breath related diseases biomarkers for chewing or mastication, biomarkers for digestive disorders, biomarkers for hepatic diseases, spleen, gall-bladder and pancreatic diseases, biomarkers for urinary system disorders, biomarkers for integumentary system diseases, biomarkers for endocrine, biomarkers for Cushing disease, biomarkers for Cystic Fibrosis, lymphatic, and excretory diseases, sensors which measure a cell surface component or a cellular marker or component, sensors which measure a pathogen or a microbe, sensors which measure administered foreign materials, medications, diagnostic molecules, drugs, biologically sensitive, derived, bio-mimics, or bioengineered molecules, sensors which measure an ingested molecule or its metabolite, including wherein ingested molecule is a pathogen, a microbial, an ingested toxin, an ingested allergen, an ingested food constituent, including a nutrient, a micronutrient, a fat molecule, a carbohydrate molecule, a sugar molecule, a protein molecule, an amino acid, sensors which measure ingested medications, ingested foreign material, ingested drugs, an ingested diagnostic molecule, an ingested biologically sensitive molecule, an ingested nanoparticle, an ingested derived molecule, a biomimic, an ingested bioengineered molecule, a disease-related biomarker related to a disease diagnosable by a sensors which includes or consists of sensors of blood pressure, core body temperature, heart rate, levels of a predetermined biologic, chemical or medication or their metabolites.

5. The device of claim 2 wherein the at least one part of transmucosal extraction device is configured to contain at least one permeable membrane, semi-permeable membrane, micro-pump, nano-pump, paper microfluidics, milli-fluidics module, microfluidics module, nanofluidics chip or module, permeable membrane, semi-permeable membrane, non-permeable, synthetic membranes, biological membranes, coated membranes, uncoated membranes, color changing membranes, microfiltration membrane, ultra-filtration membranes, nano-filtration membranes, osmotic membranes, reverse osmotic membranes, ionic or charged membranes, neutral membranes, size-exclusion membranes, affinity membranes, adsorption membranes, extracting membranes, binding membranes, hydrophilic membranes, hydrophobic membranes, electrostatic attraction and repulsing membranes, polar membranes, non-polar membranes, lipophillic membranes, lipophobic membranes, coated membranes, resistant membranes, capillary action membranes, suction generating membranes, micro-pumps, absorbent materials, pressure generating membranes, surface tension generating membranes, energy releasing membranes, energy absorbing membranes, and reactive membranes, coated micro- or nano-beads, non-coated micro-, nano-beads or gel.

6. The device of claim 1 wherein the receptacle is selected from the group consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, elderly, and animals, and the receptacle is a infant, children, adult, and animal pacifier, pacifier like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible transmucosal extraction patch, stretch stripes, adhesive stripes, transmucosal patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick;
horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a smart gauge, smart dipstick, smart rod, smart stick, biteplate, bendable or flexible sensors and transmucosal extraction device unit, electronic insert, stretch stripes, adhesive stripes, transmucosal patches or tattoos; oral surgical, medical devices, and hospitalization equipment function, anesthetic machine for continuous-flow, vaporizer, oxygen mask, nasal oxygen mask, Guedel airways, oral Suction catheter, ventilation, respiratory maintenance circuit, Laryngoscope, Tracheal tube, Laryngeal mask airway, tracheostomy tube, endoscopes, eschmann stylet or gum elastic bougie, air-filters, mucus sucker, an attachment to a tooth, inner-lips, or inner-cheeks, inner lip clip, inner cheek clip, an insert in a gum, gum-cover, a smart gauge, smart dipstick, smart rod, smart stick, thermometer, a cap, a crown, a bridge, a guard, a denture, a dental implant, a veneer, a filling, a fixed prosthesis, a brace, a dental wire, a retainer, a tongue piercing, and any combination thereof.

7. The device of claim 1 wherein the receptacle is securely placed within the oral cavity of an animal or human comprises one or more of being inserted, implanted, attached, or implantable fixedly inserted, imbedded, fitted, fixed, implanted, fastened, joined, associated, coupled, linked, banded, united, suction, capillary, clipped, mounted, combined, glued, adhered, cemented, or firmly connected by mouth parts, onlays or inlays, moldable materials, materials embedded, cemented or adhered to a palate, inside of cheeks, lips, tongue, sublingual cavity, gums, jaw, teeth, oral tissue, oral muscles, or oral teeth, oral bones, and any combination thereof.

8. The device of claim 1 wherein the receptacle is securely placed by micro- and nano-clips, frames, brackets, sealants, dental composites, bonds, adhesives, adhesive strips, cements, wires, bands, glues, embedment, injection, printing, tattooing, or any combination thereof.

9. The device of claim 1 wherein the said device is configured to be at least one interface with a network configured to utilize the information obtained from one or more extraction devices or from the one or more sensors or from one or more platforms.

10. The device of claim 1 wherein the at least one part of the said device is configured to be manually or automatically turned on or off.

11. The device of claim 1 wherein the at least one part of the said device is configured to be removable and installable.

12. The device of claim 1 wherein a micro-pump is configured to control suction of blood, interstitial fluid or its' components into various chambers.

13. The device of claim 1 wherein the said device is configured to couple to an oral sensor wearable technologies (OSWT)-transmucosal extraction system which can be coupled to communication systems and alerting technology that links a multitude of transmucosal extraction information inputs together.

14. The device of claim 1 wherein the said receptor is configured to collect, concentrates, isolate, and store blood or interstitial fluid components.

15. The device of claim 1 wherein the said removal part is configured to be read by an extra-oral reader device.

16. The device of claim 15 wherein the reader device further comprising one or more sensors contained within or upon the reader, and at least one interface with a network configured to utilize the information obtained from the one or more sensors or from one or more platforms.

17. The device of claim 15 wherein the device further comprising of at least one biosensor interacts with at least one disease-related biomarkers from blood, interstitial fluid, lymph node, bone, or tooth constituent selected from the group consisting of oral and systemic infectious diseases, cancers, drug abuse, metabolic diseases, malnutrition, obesity, cardiovascular diseases, atherosclerotic inflammation, stroke measure a predetermined biologic, measure a predetermined biologic including or consisting of DNA, RNA, telomeres, methylated or otherwise modified DNA or RNA, proteins, immunoglobins, antibodies, histones, peptides, modified proteins, neuro-peptides, pigments, and enzymes, measure dissolved gases, including oxygen, carbon dioxide, carbon monoxide, ammonia, sulphur, an alcohol-containing gas, measure a lipids profile, measure a chemical molecule, measure a salt, an alcohol, a metabolite, an anion, a cation, water, a sugar, a protein, a lectin, measure a drug, a medication, measure cells, cancerous cells, biomarkers for drug abuse, biomarkers for a metabolic disease, biomarkers for malnutrition, biomarkers for obesity, biomarkers for a cardiovascular disease, biomarkers for atherosclerotic, biomarkers for infection, biomarkers for auto-immune, other immune diseases, biomarkers for stroke, biomarkers for AIDs, biomarkers for multiple sclerosis, biomarkers for periodontal diseases, biomarkers for brain-function disorders, dementia, memory loss, depression, mental disease, Alzheimer's disease, mentally-challenged disorders, nervous system disorders, tracking or wandering, and other psychology and neurological disorders, biomarkers for bleeding, head and neck injuries, biomarkers for Sjogen's syndrome, biomarkers for oxidative stress, biomarkers for allergies, biomarkers for cancer, biomarkers for skeletal and muscle diseases, biomarkers for genetic diseases, biomarkers for renal diseases, biomarkers for osteoporosis, biomarkers for fatigue, biomarkers for stress, biomarkers for sleep deprivation or sleep apnea, biomarkers for fertility, pregnancy, ovulation, and reproductive system disorders, biomarkers for cystic fibrosis, biomarkers for respiratory or pulmonary diseases, biomarkers for diabetes and ketoacidosis, biomarkers for inflammation, biomarkers for age-related diseases, biomarkers for dehydration, biomarkers for halitosis, biomarkers for alcohol consumption, alcoholism or drug consumption or drug addiction, biomarkers for hypoxia, smoking-related diseases, toxins, or pollutants, biomarkers for poor-gait, biomarkers for Crohn's disease, biomarkers for dental caries, biomarkers for blood and circulatory disorders, biomarkers for ear, nose, and throat diseases, biomarkers for taste, Ageusia, Hypogeusia, or Dysgeusia, biomarkers for bad-breath related diseases biomarkers for chewing or mastication, biomarkers for digestive disorders, biomarkers for hepatic diseases, spleen, gall-bladder and pancreatic diseases, biomarkers for urinary system disorders, biomarkers for integumentary system diseases, biomarkers for endocrine, biomarkers for Cushing disease, biomarkers for Cystic Fibrosis, lymphatic, and excretory diseases, sensors which measure a cell surface component or a cellular marker or component, sensors which measure a pathogen or a microbe, sensors which measure administered foreign materials, medications, diagnostic molecules, drugs, biologically sensitive, derived, bio-mimics, or bioengineered molecules, sensors which measure an ingested molecule or its metabolite, including wherein ingested molecule is a pathogen, a microbial, an ingested toxin, an ingested allergen, an ingested food constituent, including a nutrient, a micronutrient, a fat molecule, a carbohydrate molecule, a sugar molecule, a protein molecule, an amino acid, sensors which measure ingested medications, ingested foreign material, ingested drugs, an ingested diagnostic molecule, an ingested biologically sensitive molecule, an ingested nanoparticle, an ingested derived molecule, a biomimic, an ingested bioengineered molecule, a disease-related biomarker related to a disease diagnosable by a sensors which includes or consists of sensors of blood pressure, core body temperature, heart rate, levels of a predetermined biologic, chemical or medication or their metabolites.

18. The device of claim 15 further comprising an interface with at least one biosensor not located within the animal's oral cavity.

19. The device of claim 15 wherein the said reader is configured to “read” biological information using one or more sensors selected from the group consisting of, but not limited to, contact, non-contact temperature, infrared, pressure of gas or liquid, absolute pressure differential pressure, vacuum pressure, gauge pressure, conductive rubber pressure, lead zirconate titanate pressure, Polyvinylidene fluoride pressure, PVDF-TrFE pressure, FETs pressure, metallic capacitive sensing elements pressure, resistance, tactile, elasto resistive sensors, conductivity, color, luminance, movement, optical, photo sensors, photo detectors, pixel (a light sensor and an active amplifier), light dependent resistors, optical filters, fluorescence, phosphorescence, sound, resonant, humidity, changes in humidity (measurements of mass, a mechanical, electrical changes as moisture is absorbed, could be used to measure humidity, or changes in temperature of condensation, changes in electrical capacitance, resistance, dielectric constant, dew point hygrometer, thermal conductivity temperature, or thermal conductivity), light, magnetic, electromagnetic, position, ionization, pH measurements, electrodes, fundamental electrical measurements, piezo-electric, piezo-resistive, potentiometric, orientation, video, 2 or 3D images, density, mass, MEMS, Lab-On-Chip to Micro Total Analysis, biosensors, chemo-sensors, biologic-sensor, a biologically-relevant-sensor, temperature, blood chemicals, blood electrolytes, pH, blood oxygen level, respired gases, gases, optimum breathing, oral air-flow, gyroscopic measurement, accelerometer measurement 1D, accelerometer measurement 2D, accelerometer measurement 3D, kinematics, ionic conductivity, photos, videos, images, electrical waves, sound waves, spectrophotometry, electromagnetic spectrum, gamma waves, X-ray wave, ultraviolet waves, visible waves, infrared waves, terahertz waves, microwaves, radio waves, magnetic waves, ultrasonic waves, magnetic resonance, magnetic field, electro- or magnetic-encephalography, functional magnetic resonance imaging, optical topography, global positioning or tracking, and radiation wave activity.

20. The device of claim 15 wherein at least a part of the said reader is configured to connect to a smart phone such that the light source on the cellular phone could be converted to excitation energy source, the camera could be converted to a light measuring device such as optical photo, density, or colorimeter, video, photodiode, LED, the touch sensors or pressure sensors in the cellular phone could be used to measure changes in volume, pressure, other cellular phone functions could be used to measure, record, diagnose, and changes in biological information.
